(19) United States
(12) Patent Application Publication
(10) Pub. No.: US 2020/0256880 A1
(43) Pub. Date: Aug. 13, 2020

(54) Neuronal Assay Method Involving Calcineurin

(71) Applicant: The Broad Institute, Inc., Cambridge, MA (US)

(72) Inventors: Li Li, Arlington, MA (US); Yan-Ling Zhang, Lexington, MA (US); Ayan Ghoshal, Somerville, MA (US); Jeffrey Cottrell, Sterling, MA (US)

(21) Appl. No.: 16/639,251

(22) PCT Filed: Aug. 16, 2018
(86) PCT No.: PCT/IB2018/056171

§ 371 Date: Feb. 14, 2020

Related U.S. Application Data
(60) Provisional application No. 62/546,464, filed on Aug. 16, 2017.

(51) Int. Cl. G01N 33/68; G01N 33/52; C07K 16/18

(52) U.S. Cl. CPC G01N 33/6896; C07K 2317/565; C07K 16/18; G01N 33/52

(57) ABSTRACT

This disclosure relates to methods of detecting and quantifying neuronal activation. This disclosure further relates to a particular phosphorylation of calcineurin A alpha (CNAα) at serine 469 (S469) of the amino acid sequence of the protein as a marker for neuronal activation, antibodies recognizing that phosphorylated form of CNAα, and uses of such antibodies, for example, in various neuronal activity assays and related kits.

This disclosure relates to methods of detecting and quantifying neuronal activation. This disclosure further relates to a particular phosphorylation of calcineurin A alpha (CNAα) at serine 469 (S469) of the amino acid sequence of the protein as a marker for neuronal activation, antibodies recognizing that phosphorylated form of CNAα, and uses of such antibodies, for example, in various neuronal activity assays and related kits.

### BACKGROUND

The activity of neurons in a network is an important means to assess neurologic parameters, such as excitability and seizure potential. Neuronal activity may comprise various parameters such as excitability of individual neurons, level of interneuronal network activity, communication between neurons, and synaptogenesis. Thus, measuring the activity of neurons may be critical for understanding synaptic transmission and the biologic basis of neurologic disorders and for understanding how neuronal activity can be impacted by genetic mutations or modified by therapeutic agents.

However, traditional methods of assessing neuronal activity are time-intensive and low-throughput. For example, electrophysiological measures of neuronal activity require a high-degree of expertise and specialized equipment that may not be available to all laboratories that would be interested in studying neuronal activity. Immunohistochemistry (IHC) and in situ hybridization (ISH) assays of cultured neurons are also available, but may track neuronal activity through analysis of RNA or protein expression such as of immediate early proteins. There is a time lag between activation of neurons and the expression of these RNAs or proteins, which can make such assays lengthy to perform and potentially not reflective of transient changes in neuronal activity in response to various stimuli. As such, a relatively high throughput and easy to use assay that measures neuronal activity in close to real time may be quite useful in diagnostic and screening protocols.

The present inventors have found that the level of neuronal activity can be detected in closer to real time by measuring neuronal activity-dependent phosphorylation at position S469 of calcineurin A alpha (CNAα) as a marker.

Further, the inventors have found that antibodies specifically recognizing phospho-S469 CNAα allow for sensitive measurement of changes in neuronal activity. These phosphospecific antibodies may be used in various neuronal activity screening systems and in various diagnostic assays of neurologic disorders and associated therapeutics, and as part of kits that may be used to perform such assays.

### SUMMARY

The present disclosure provides, inter alia, methods for measuring neuronal activity in which the level of phosphorylation of S469 of CNAα is used as a marker for neuronal activation. For example, a specific antibody or aptamer may be used to recognize the phosphorylation of S469 of the protein.

Methods herein include methods comprising, for example obtaining a tissue sample comprising neurons or a sample comprising cultured neurons; exposing the sample to one or more chemical compounds or physical signals that may alter activity of neurons in the sample; exposing the sample to a phospho-S469 CNAα antibody; optionally washing the sample one or more times in between exposing the sample to these reagents; and detecting the level of phospho-S469 CNAα antibody retained by the sample, wherein the level of phospho-S469 CNAα antibody retained by the sample indicates the neuronal activity level in the sample. Also provided herein are methods for measuring the level of phospho-S469 CNAα antibody retained by a tissue sample comprising neurons or a sample comprising cultured neurons, the method comprising: obtaining a tissue sample comprising neurons or a sample comprising cultured neurons; exposing the sample to one or more chemical compounds or physical signals that may alter activity of neurons in the sample; exposing the sample to the phospho-S469 CNAα antibody; optionally washing the sample one or more times in between the exposing and detecting steps; and detecting the level of phospho-S469 CNAα antibody retained by the sample. In some exemplary methods, the sample is exposed to a phospho-S469 CNAα antibody for 1 to 24 hours at a temperature of between 0 and 40° C., such as for 12-24 hours, 16-20 hours, 16, 17, 18, 19, or 20 hours at 0 to 10° C., e.g. at 3, 4, 5, 6, 7, or 8° C., or for a shorter time at higher temperatures, such as 1-4 hours, such as 1-2, or 2-3 hours at 20-40° C., such as 20-30° C., 22-27° C., 22, 23, 24, 25, 26, or 27° C. In some embodiments, the level of phospho-S469 CNAα antibody retained by the sample is detected by fluorescence or chemiluminescence staining. In some embodiments, the level of phospho-S469 CNAα antibody retained by the sample is detected in less than 30 minutes, less than 15 minutes, less than 10 minutes, or less than 5 minutes after exposure of the sample to the phospho-S469α antibody, for example within 1-30 minutes, 1-20 minutes, 1-15 minutes, 1-10 minutes, 1-5 minutes, or 5-10 minutes after exposure. In some embodiments, the methods comprise determining chemical long term potentiation (cLTP) of neurons in the sample from exposure to the one or more chemical compounds, wherein the level of phospho-S469 CNAα antibody retained by the sample indicates the cLTP. In some embodiments, the sample comprises cultured neurons and the cultured neurons are derived from induced pluripotent stem cells (iPSCs) or from mammalian hippocampal, cortical, striatum, cerebellar granule, or other mammalian neuronal cells. In some embodiments, the sample is a brain tissue sample. In some embodiments, the methods use a phospho-S469 CNAα antibody that comprises a heavy chain and a light chain, wherein the heavy chain comprises a heavy chain complementary determining region 1 (HCDR1) comprising the amino acid sequence of SEQ ID NO: 11; a HCDR2 comprising the amino acid sequence of SEQ ID NO: 12; and a HCDR3 comprising the amino acid sequence of SEQ ID NO: 13; and wherein the light chain comprises a light chain complementary determining region 1 (LCDR1) comprising the amino acid sequence of SEQ ID NO: 14; a LCDR2 comprising the amino acid sequence of SEQ ID NO: 15; and a LCDR3 comprising the amino acid sequence of SEQ ID NO: 16. In some embodiments, the heavy chain of the antibody comprises a heavy chain variable region with an amino acid sequence that is at least 90%, at least 95%, at least 97%, at least 98%, or at least 99% identical to the amino acid sequence of SEQ ID NO: 7. In some embodiments the light chain of the antibody comprises a light chain variable region amino acid sequence that is at least 90%, at least 95%, at least 97%, at least 98%, or at least 99% identical to the amino acid sequence of SEQ ID NO: 9. In some embodiments, the heavy chain variable region of the antibody comprises the amino acid sequence of SEQ ID NO: 7. In some embodiments, the light chain variable region of the antibody comprises the amino acid sequence of SEQ ID NO: 9. In some embodiments, the heavy chain variable region of the antibody comprises the amino acid sequence of SEQ ID NO: 7 and the light chain variable region of the antibody comprises the amino acid sequence of SEQ ID NO: 9.

Also provided herein are methods for identifying chemical compounds that increase or suppress neuronal activity comprising: obtaining a tissue sample comprising neurons or a sample comprising cultured neurons; exposing the sample to one or more chemical compounds that may alter activity of neurons in the sample; exposing the sample to a phospho-S469 CNAα antibody; and detecting the level of phospho-S469 CNAα antibody retained by the sample, wherein the level of phospho-S469 CNAα antibody retained by the sample indicates the neuronal activity level in the sample and whether the one or more chemical compounds alters the activity of neurons in the sample. Again, optionally, one or more wash steps are performed after exposing the sample to the one or more chemical compounds and/or to the antibody and prior to detection of the retained antibody. In some exemplary methods, the sample is exposed to a phospho-S469 CNAα antibody for 1 to 24 hours at a temperature of between 0 and 40° C., such as for 12-24 hours, 16-20 hours, 16, 17, 18, 19, or 20 hours at 0 to 10° C., e.g. at 3, 4, 5, 6, 7, or 8° C., or for a shorter time at higher temperatures, such as 1-4 hours, such as 1-2, or 2-3 hours at 20-40° C., such as 20-30° C., 22-27° C., 22, 23, 24, 25, 26, or 27° C. In some embodiments, the level of phospho-S469 CNAα antibody retained by the sample is detected by fluorescence or chemiluminescence staining. In some embodiments, the level of phospho-S469 CNAα antibody retained by the sample is detected in less than 30 minutes, less than 15 minutes, less than 10 minutes, or less than 5 minutes after exposure of the sample to the phospho-S469α antibody, for example within 1-30 minutes, 1-20 minutes, 1-15 minutes, 1-10 minutes, 1-5 minutes, or 5-10 minutes after exposure. In some embodiments, the methods comprise determining chemical long term potentiation (cLTP) of neurons in the sample from exposure to the one or more chemical compounds, wherein the level of phospho-S469 CNAα antibody retained by the sample indicates the cLTP. In some embodiments, the sample comprises cultured neurons and the cultured neurons are derived from induced pluripotent stem cells (iPSCs) or from mammalian hippocampal, cortical, striatum, cerebellar granule, or other mammalian neuronal cells. In some embodiments, the sample is a brain tissue sample. In some embodiments, the sample is an iPSC sample from a human patient with a neurological disorder. In some embodiments, the iPSC sample is from a patient with autism, epilepsy, fragile X syndrome, or cerebral ischemia. In some embodiments, the sample is a mammalian tissue sample from a model for schizophrenia, bipolar disorder, epilepsy, Alzheimer's disease, alcoholic Korsakoff disease (KS), multiple sclerosis, Parkinson's disease, Down's syndrome, Williams syndrome, Specific Language Impairment, and Attention Deficit Hyperactivity Disorder (ADHD), spinal cord injury, stroke, traumatic brain injury, hearing loss, neurodegenerative diseases such as amyotrophic lateral sclerosis (ALS), Huntington's disease, epilepsy, autism, fragile X syndrome, alcoholism, alcohol withdrawal, benzodiazepine withdrawal, or hypoglycemia. In some embodiments, the sample is a tissue sample sample from a human patient with a neurological disorder or a tissue sample derived from an animal model of a neurological disorder. In some such embodiments, the tissue sample acts as a model for human neurological disorders such as schizophrenia, bipolar disorder, epilepsy, Alzheimer's disease, alcoholic Korsakoff disease (KS), multiple sclerosis, Parkinson's disease, Down's syndrome, Williams syndrome, Specific Language Impairment, and Attention Deficit Hyperactivity Disorder (ADHD), spinal cord injury, stroke, traumatic brain injury, hearing loss, neurodegenerative diseases such as amyotrophic lateral sclerosis (ALS), Huntington's disease, epilepsy, autism, fragile X syndrome, alcoholism, alcohol withdrawal, benzodiazepine withdrawal, or hypoglycemia. In some embodiments, the methods use a phospho-S469 CNAα antibody that comprises a heavy chain and a light chain, wherein the heavy chain comprises a heavy chain complementary determining region 1 (HCDR1) comprising the amino acid sequence of SEQ ID NO: 11; a HCDR2 comprising the amino acid sequence of SEQ ID NO: 12; and a HCDR3 comprising the amino acid sequence of SEQ ID NO: 13; and wherein the light chain comprises a light chain complementary determining region 1 (LCDR1) comprising the amino acid sequence of SEQ ID NO: 14; a LCDR2 comprising the amino acid sequence of SEQ ID NO: 15; and a LCDR3 comprising the amino acid sequence of SEQ ID NO: 16. In some embodiments, the heavy chain of the antibody comprises a heavy chain variable region with an amino acid sequence that is at least 90%, at least 95%, at least 97%, at least 98%, or at least 99% identical to the amino acid sequence of SEQ ID NO: 7. In some embodiments the light chain of the antibody comprises a light chain variable region amino acid sequence that is at least 90%, at least 95%, at least 97%, at least 98%, or at least 99% identical to the amino acid sequence of SEQ ID NO: 9. In some embodiments, the heavy chain variable region of the antibody comprises the amino acid sequence of SEQ ID NO: 7. In some embodiments, the light chain variable region of the antibody comprises the amino acid sequence of SEQ ID NO: 9. In some embodiments, the heavy chain variable region of the antibody comprises the amino acid sequence of SEQ ID NO: 7 and the light chain variable region of the antibody comprises the amino acid sequence of SEQ ID NO: 9.

Also provided herein are methods for determining a personalized therapy for a subject having a neurological disorder, comprising: obtaining a tissue sample comprising neurons, a sample comprising cultured neurons derived from iPSCs derived from the subject, or a sample comprising cultured neurons from the subject; exposing the sample to one or more therapeutic agents for the neurological disorder; exposing the sample to a phospho-S469 CNAα antibody; optionally performing one or more wash steps after the exposing steps and prior to detection; and detecting the level of phospho-S469 CNAα antibody retained by the sample, wherein the level of phospho-S469 CNAα antibody retained by the sample indicates the neuronal activity level in the sample and whether one or more therapeutic agents alters the activity of neurons in the subject's sample. In some embodiments, the patient has a neurological disorder as described herein below. In some embodiments, the neurological disorder is schizophrenia, bipolar disorder, epilepsy, Alzheimer's disease, alcoholic Korsakoff disease (KS), multiple sclerosis, Parkinson's disease, Down's syndrome, Williams syndrome, Specific Language Impairment, and Attention Deficit Hyperactivity Disorder (ADHD), spinal cord injury, stroke, traumatic brain injury, hearing loss, neurodegenerative diseases such as amyotrophic lateral sclerosis (ALS), Huntington's disease, epilepsy, autism, fragile X syndrome, alcoholism, alcohol withdrawal, benzodiazepine withdrawal, or hypoglycemia. In some embodiments, the neurological disorder is epilepsy, autism, fragile X syndrome, or cerebral ischemia. In some embodiments, the subject is treated with one or more therapeutic agents shown by the method to increase neuronal activity level in the sample. In some embodiments, the methods comprise determining chemical long term potentiation (cLTP) of neurons in the sample from exposure to the one or more therapeutic agents, wherein the level of phospho-S469 CNAα antibody retained by the sample indicates the cLTP. In some embodiments, the subject is treated with one or more therapeutic agents shown to increase neuronal activity or cLTP of neurons in the subject's sample. In some exemplary methods, the sample is exposed to a phospho-S469 CNAα antibody for 1 to 24 hours at a temperature of between 0 and 40° C., such as for 12-24 hours, 16-20 hours, 16, 17, 18, 19, or 20 hours at 0 to 10° C., e.g. at 3, 4, 5, 6, 7, or 8° C., or for a shorter time at higher temperatures, such as 1-4 hours, such as 1-2, or 2-3 hours at 20-40° C., such as 20-30° C., 22-27 ° C., 22, 23, 24, 25, 26, or 27° C. In some embodiments, the level of phospho-S469 CNAα antibody retained by the sample is detected by fluorescence or chemiluminescence staining. In some embodiments, the level of phospho-S469 CNAα antibody retained by the sample is detected in less than 30 minutes, less than 15 minutes, less than 10 minutes, or less than 5 minutes after exposure of the sample to the phospho-S469α antibody, for example within 1-30 minutes, 1-20 minutes, 1-15 minutes, 1-10 minutes, 1-5 minutes, or 5-10 minutes after exposure. In some embodiments, the methods use a phospho-S469 CNAα antibody that comprises a heavy chain and a light chain, wherein the heavy chain comprises a heavy chain complementary determining region 1 (HCDR1) comprising the amino acid sequence of SEQ ID NO: 11; a HCDR2 comprising the amino acid sequence of SEQ ID NO: 12; and a HCDR3 comprising the amino acid sequence of SEQ ID NO: 13; and wherein the light chain comprises a light chain complementary determining region 1 (LCDR1) comprising the amino acid sequence of SEQ ID NO: 14; a LCDR2 comprising the amino acid sequence of SEQ ID NO: 15; and a LCDR3 comprising the amino acid sequence of SEQ ID NO: 16. In some embodiments, the heavy chain of the antibody comprises a heavy chain variable region with an amino acid sequence that is at least 90%, at least 95%, at least 97%, at least 98%, or at least 99% identical to the amino acid sequence of SEQ ID NO: 7. In some embodiments the light chain of the antibody comprises a light chain variable region amino acid sequence that is at least 90%, at least 95%, at least 97%, at least 98%, or at least 99% identical to the amino acid sequence of SEQ ID NO: 9. In some embodiments, the heavy chain variable region of the antibody comprises the amino acid sequence of SEQ ID NO: 7. In some embodiments, the light chain variable region of the antibody comprises the amino acid sequence of SEQ ID NO: 9. In some embodiments, the heavy chain variable region of the antibody comprises the amino acid sequence of SEQ ID NO: 7 and the light chain variable region of the antibody comprises the amino acid sequence of SEQ ID NO: 9.

The disclosure herein also provides methods of treating a subject with a neurological disorder, comprising: (A) obtaining results from a method as described above performed in a sample from the subject, and (B) administering to the subject one or more therapeutic agents determined from the results to increase the activity of neurons in the subject's sample. In some embodiments, the methods use a phospho-S469 CNAα antibody that comprises a heavy chain and a light chain, wherein the heavy chain comprises a heavy chain complementary determining region 1 (HCDR1) comprising the amino acid sequence of SEQ ID NO: 11; a HCDR2 comprising the amino acid sequence of SEQ ID NO: 12; and a HCDR3 comprising the amino acid sequence of SEQ ID NO: 13; and wherein the light chain comprises a light chain complementary determining region 1 (LCDR1) comprising the amino acid sequence of SEQ ID NO: 14; a LCDR2 comprising the amino acid sequence of SEQ ID NO: 15; and a LCDR3 comprising the amino acid sequence of SEQ ID NO: 16. In some embodiments, the heavy chain of the antibody comprises a heavy chain variable region with an amino acid sequence that is at least 90%, at least 95%, at least 97%, at least 98%, or at least 99% identical to the amino acid sequence of SEQ ID NO: 7. In some embodiments the light chain of the antibody comprises a light chain variable region amino acid sequence that is at least 90%, at least 95%, at least 97%, at least 98%, or at least 99% identical to the amino acid sequence of SEQ ID NO: 9. In some embodiments, the heavy chain variable region of the antibody comprises the amino acid sequence of SEQ ID NO: 7. In some embodiments, the light chain variable region of the antibody comprises the amino acid sequence of SEQ ID NO: 9. In some embodiments, the heavy chain variable region of the antibody comprises the amino acid sequence of SEQ ID NO: 7 and the light chain variable region of the antibody comprises the amino acid sequence of SEQ ID NO: 9.

This disclosure also provides isolated antibodies that specifically bind to phospho-S469 CNAα. In some embodiments the antibodies specifically bind to an epitope comprising the sequence of SEQ ID NO: 1. In some embodiments, the antibodies do not specifically bind to an epitope that: (a) comprises the sequence of SEQ ID NO:2 but (b) does not comprise the sequence of SEQ ID NO:1. In some embodiments, the antibodies do not specifically bind to an epitope consisting of the sequence of SEQ ID NO:2. In some embodiments, the epitope comprising the sequence of SEQ ID NO: 1 comprises no more than 10 amino acids upstream of the N-terminus of SEQ ID NO: 1 and no more than 5 amino acids downstream of the C-terminus of SEQ ID NO: 1. In some embodiments, the antibodies specifically bind to an epitope consisting of the sequence of SEQ ID NO: 1. In some embodiments, the antibodies have one or more of the following properties: do not specifically bind to either CNAβ or CNAγ; do not specifically bind to an epitope comprising SEQ ID NO: 4; do not specifically bind to an epitope comprising SEQ ID NO: 5; do not specifically bind to an epitope comprising SEQ ID NO: 6; do not specifically bind to CNAα domains other than the autoinhibitory domain; and bind to murine or cynomolgus phospho-S469 CNAα. In some embodiments, the antibody comprises a heavy chain and a light chain, wherein the heavy chain comprises a heavy chain complementary determining region 1 (HCDR1) comprising the amino acid sequence of SEQ ID NO: 11; a HCDR2 comprising the amino acid sequence of SEQ ID NO: 12; and a HCDR3 comprising the amino acid sequence of SEQ ID NO: 13; and wherein the light chain comprises a light chain complementary determining region 1 (LCDR1) comprising the amino acid sequence of SEQ ID NO: 14; a LCDR2 comprising the amino acid sequence of SEQ ID NO: 15; and a LCDR3 comprising the amino acid sequence of SEQ ID NO: 16. In some embodiments, the heavy chain of the antibody comprises a heavy chain variable region with an amino acid sequence that is at least 90%, at least 95%, at least 97%, at least 98%, or at least 99% identical to the amino acid sequence of SEQ ID NO: 7. In some embodiments the light chain of the antibody comprises a light chain variable region amino acid sequence that is at least 90%, at least 95%, at least 97%, at least 98%, or at least 99% identical to the amino acid sequence of SEQ ID NO: 9. In some embodiments, the heavy chain variable region of the antibody comprises the amino acid sequence of SEQ ID NO: 7. In some embodiments, the light chain variable region of the antibody comprises the amino acid sequence of SEQ ID NO: 9. In some embodiments, the heavy chain variable region of the antibody comprises the amino acid sequence of SEQ ID NO: 7 and the light chain variable region of the antibody comprises the amino acid sequence of SEQ ID NO: 9.

In some embodiments, the antibodies are Fv, scFv, Fab, Fab′, (Fab′)2 or other antigen binding fragments. In some embodiments, the antibodies are multispecific or bispecific or diabodies, triabodies, or tetrabodies, or are humanized, chimeric, mouse, or human. In some embodiments, the antibodies further comprise a detection reagent comprising at least one of a bead, a fluorescent label, a chemiluminescent label, a secondary antibody, or a tertiary antibody. For example, the antibodies may be covalently conjugated to such a reagent either directly or through a linker, for example, or they may be non-covalently bound to such a reagent.

Also provided herein are methods of obtaining a phospho-S469 CNAα antibody such as described above, comprising exposing a laboratory animal to an epitope comprising the sequence of SEQ ID NO: 1 and selecting for antibodies made by the animal that specifically bind to the epitope. In some embodiments, the methods further comprise selecting for an antibody made by the animal that has one or more of the following properties: specifically binds to an epitope consisting of the sequence of SEQ ID NO: 1; does not specifically bind to an epitope that: (a) comprises the sequence of SEQ ID NO:2 but (b) does not comprise the sequence of SEQ ID NO:1; does not specifically bind to an epitope consisting of the sequence of SEQ ID NO:2; does not specifically bind to either CNAβ or CNAγ does not specifically bind to an epitope comprising SEQ ID NO: 4; does not specifically bind to an epitope comprising SEQ ID NO: 5; does not specifically bind to an epitope comprising SEQ ID NO: 6; does not specifically bind to CNAα domains other than the autoinhibitory domain; and binds to murine or cynomolgus phospho-S469 CNAα. In some embodiments, the antibody comprises a heavy chain and a light chain, wherein the heavy chain comprises a heavy chain complementary determining region 1 (HCDR1) comprising the amino acid sequence of SEQ ID NO: 11; a HCDR2 comprising the amino acid sequence of SEQ ID NO: 12; and a HCDR3 comprising the amino acid sequence of SEQ ID NO: 13; and wherein the light chain comprises a light chain complementary determining region 1 (LCDR1) comprising the amino acid sequence of SEQ ID NO: 14; a LCDR2 comprising the amino acid sequence of SEQ ID NO: 15; and a LCDR3 comprising the amino acid sequence of SEQ ID NO: 16. In some embodiments, the heavy chain of the antibody comprises a heavy chain variable region with an amino acid sequence that is at least 90%, at least 95%, at least 97%, at least 98%, or at least 99% identical to the amino acid sequence of SEQ ID NO: 7. In some embodiments the light chain of the antibody comprises a light chain variable region amino acid sequence that is at least 90%, at least 95%, at least 97%, at least 98%, or at least 99% identical to the amino acid sequence of SEQ ID NO: 9. In some embodiments, the heavy chain variable region of the antibody comprises the amino acid sequence of SEQ ID NO: 7. In some embodiments, the light chain variable region of the antibody comprises the amino acid sequence of SEQ ID NO: 9. In some embodiments, the heavy chain variable region of the antibody comprises the amino acid sequence of SEQ ID NO: 7 and the light chain variable region of the antibody comprises the amino acid sequence of SEQ ID NO: 9.

Also provided herein are kits for measuring the level of phospho-S469 CNAα in a biological sample, wherein the kit comprises phospho-S469 CNAα antibodies and one or more reagents for detecting the level of phospho-S469 CNAα antibodies in the biological sample. In some embodiments, the kits further comprise at least one chemical compound that may alter neuronal activity in a biological sample comprising neurons as a positive or negative control, whereby exposing the phospho-S469 CNAα antibody and the at least one chemical compound to a biological sample comprising neurons to allows for determination of the effect of the at least one chemical compound on activity of the neurons in the sample. In some kits herein, the phospho-S469 CNAα antibodies are conjugated to beads. In some kits herein, the reagents for detecting the level of phospho-S469 CNAα antibodies in the biological sample comprise fluorescent or chemiluminescent labels. In some kits herein, the reagents for detecting the level of phospho-S469 CNAα antibodies in the biological sample comprise secondary antibodies and optionally tertiary antibodies. In some embodiments, the kits further comprise instructions for use. In some embodiments, the kits further comprise a substrate for holding a biological sample, such as a multi-well plate. In some embodiments, the kits comprise a phospho-S469 CNAα antibody that comprises a heavy chain and a light chain, wherein the heavy chain comprises a heavy chain complementary determining region 1 (HCDR1) comprising the amino acid sequence of SEQ ID NO: 11; a HCDR2 comprising the amino acid sequence of SEQ ID NO: 12; and a HCDR3 comprising the amino acid sequence of SEQ ID NO: 13; and wherein the light chain comprises a light chain complementary determining region 1 (LCDR1) comprising the amino acid sequence of SEQ ID NO: 14; a LCDR2 comprising the amino acid sequence of SEQ ID NO: 15; and a LCDR3 comprising the amino acid sequence of SEQ ID NO: 16. In some embodiments, the heavy chain of the antibody comprises a heavy chain variable region with an amino acid sequence that is at least 90%, at least 95%, at least 97%, at least 98%, or at least 99% identical to the amino acid sequence of SEQ ID NO: 7. In some embodiments the light chain of the antibody comprises a light chain variable region amino acid sequence that is at least 90%, at least 95%, at least 97%, at least 98%, or at least 99% identical to the amino acid sequence of SEQ ID NO: 9. In some embodiments, the heavy chain variable region of the antibody comprises the amino acid sequence of SEQ ID NO: 7. In some embodiments, the light chain variable region of the antibody comprises the amino acid sequence of SEQ ID NO: 9. In some embodiments, the heavy chain variable region of the antibody comprises the amino acid sequence of SEQ ID NO: 7 and the light chain variable region of the antibody comprises the amino acid sequence of SEQ ID NO: 9.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows validation of the chemical long-term potentiation (cLTP) protocol. Mouse cortical neurons were treated with a control buffer (left lane of each blot) or with the cLTP protocol (right lane of each blot) and then probed on western blots for total, phospho-S831, and phospho-S845 of GluA1.

FIG. 2 shows a sample processing pipeline for discovery-based proteomic and phosphoproteomic profiling with precise relative quantification using mass tag labeling of peptides. bRP=basic reversed-phase; LC/MS=liquid chromatography mass spectrometry; RP=reversed-phase; TMT=tandem mass tag.

FIGS. 3A and 3B show cLTP proteome and phosphoproteome Log 2 changes for cLTP treated samples over control buffer tested samples. Waterfall plots showing the total (A) and phospho (B) proteome data at 5, 20, 40, and 60 min following cLTP induction. In all cases, each point is the average of two biological replicates. Statistically significant peptides are shown as arrows in (B). Where two arrows are joined, this indicates that significant peptides were present throughout the range between the arrows.

FIGS. 4A-4C show alpha paralog of the catalytic subunit of calcineurin (CNAα) phosphorylation on S469 following cLTP over time. (A, Top) The shaded boxes above the curves indicate the domains of the CNA paralogs, with identified phosphorylation sites shown by narrow lines between calmodulin (CaM) and autoinhibitory (AI) regions. (A, Bottom) The fold-changes of each identified phosphorylation site following cLTP in each of the three CNA paralogs following cLTP induction compared to control as a function of time. (B) Western blot data of mouse cortical neuron extracts at different time points following cLTP using a custom total CNAα antibody and a custom S469 phosphospecific antibody. (C) Quantification of S469 CNAα phosphorylation levels as a function of time from western blot and mass spectrometry analysis. Norm=normalized; spec=spectrometry.

FIGS. 5A-5C show that phosphorylation of S469 CNAα enhances enzyme function. (A, Left) High level crystal structure of calcineurin Aα. (A, Right) Magnified image of the calcineurin Aα catalytic site and its autoinhibitory domain (AID), showing the location of S469 at the N-terminal end of the AID. (B) Side view of the catalytic site and AID, showing the relative locations of S469 and D285 (Asp285). (C) Enzyme assays of wild-type and S469E recombinant CNAα/CNB1 holoenzyme, with and without the addition of calmodulin (CaM). pRII=phosphorylated RII peptide.

FIGS. 6A-6C show that phosphorylated S469 CNAα is enriched in dendritic spines at different time points. (A) Rat hippocampal neurons grown in 96-well plates were treated with the cLTP protocol and fixed and stained for phospho-S469 CNAα and MAP2 at 5, 20, 40, and 60 minutes afterwards. Arrows indicate neurons positive for staining of both phospho-S469 CNAα and synapsin. In control cells, no neurons were positive for phospho-S469 CNAα staining. Scale bar: 30 μm. (B) High magnification images of rat hippocampal neurons 5 minutes post-cLTP treatment, fixed, and stained for phospho-S469 CNAα and synapsin to mark presynaptic terminals. Arrows indicate representative areas of colocalization of staining for phospho-S469 CNAα and synapsin. Scale bar: 10 μm. (C) Preembedding immunoperoxidase electron microscopy staining of rat hippocampal neurons 5 min post-cLTP treatment. Arrows denote phospho-S469 CNAα staining. DS=dendritic spine; PT=presynaptic terminal. Scale bar: 200 nm.

FIGS. 7A-7D show that neuronal activity stimulates S469 CNAα phosphorylation. (A) Rat hippocampal neurons grown in 96-well plates were stimulated with various plasticity induction paradigms or modulators of neuronal activity levels, fixed and stained for phospho-S469 CNAα and MAP2, and imaged. (B) Quantification of phospho-S469 CNAα levels within MAP2-positive dendrites in wells shown in (A). (C) Quantification of total CNAα levels within MAP2-positive dendrites in wells shown in (A). (D) Rat hippocampal neurons were pretreated with vehicle, DNQX, nifedipine, APS, or a combination of all three (with group listed in this order from left to right in panel (D)) prior and after treatment with bicuculline. Neurons were then fixed and stained for phospho-S469 CNAα and synapsin and imaged on a Phenix® (Perkin Elmer) imaging system, a high content imaging system. Shown is the phospho-S469 CNAα levels localized to synapses marked by synapsin. Bic=bicuculine. *≤0.05 compared to control sample, **<0.01, ***<0.001, and ****<0.0001 compared to control sample. For FIG. 7D, statistics were derived by comparing the pharmacological condition with the equivalent control condition, either vehicle or bicucculine treated.

FIGS. 8A and 8B show that CNAα phosphorylation is stimulated by low frequency field stimulation in hippocampal slices. (A) Western blots of mouse hippocampal CA1 slices that were unstimulated (No Stim), stimulated with 0.05 Hz field stimuli for 4 min (Cont Stim), or stimulated with 0.05 Hz field stimuli for 4 min followed by a LTP-inducing paradigm (Cont Stim+HFS) and probed for phospho-S469 CNAα and total CNAα. (B) Quantification of the results shown in (A). Cont=control; HFS=high frequency stimulation; and Stim=stimulation. *≤0.05 compared to no stimulation sample. **≤0.01 compared to no stimulation sample.

FIGS. 9A and 9B show proposed kit for measuring phospho-S469 levels in cellular and tissue lysates. (A) Western blots of purified wild-type and S469E CNAα probed with total or phospho-S469 CNAα antibodies. In the middle blot, the phospho-S469 CNAα antibody was preincubated with a S469E mutated CNAα AID peptide. (B) Potential format for Alphalisa kit for measuring phospho-S469 CNAα levels within lysates that can be used to measure the levels of neuronal activity within the cells prior to lysis and/or fixation of the cells.

FIG. 10 shows phospho-S469 CNAα levels in neurons with knockdown of the autism-related genes with Fmr1, Syngap1, Pten, and Akap9 or control GSK3a small interfering RNAs (siRNAs) followed by treatment with water or bicuculline. Dashed lines indicate values for the control group for treatment with water or bicuculline. *≤0.05 compared to control treated with water. ***≤0.001 compared to control treated with bicuculline.

FIGS. 11A and 11B show (A) Western blot data of mouse cortical neuron extracts at different time points following cLTP using a custom total CNAα antibody and a monoclonal mouse anti-S469 phosphospecific antibody and (B) quantification of S469 CNAα phosphorylation levels as a function of time from the Western blot analysis.

FIGS. 12A-12C show (A) immunofluorescence staining of hippocampal neurons at various time-points following cLTP using a monoclonal mouse anti-S469 phosphospecific antibody and (B, C) quantification of the data. FIG. 12B shows quantification of antibody staining in synaptic regions of the hippocampal neurons, based on co-localization with a synaptic marker synapsinI. FIG. 12C shows quantification of antibody staining in dendritic regions of hippocampal neurons based on co-localization with dendritic marker MAP2.

FIG. 13 shows binding of CNAα by a monoclonal murine anti-S469 phosphospecific antibody following lambda phosphatase treatment of the protein with or without cLTP treatment (top lanes) and binding of CNAα by a phosphorylation nonspecific anti-CNAα antibody to show total CNAα level (bottom lanes). The data show that lambda phosphatase incubation eliminated phosphospecific antibody binding (compare left top lanes to right top lanes).

FIG. 14 provides Western blots showing that a monoclonal murine anti-S469 CNAα phosphospecific antibody does not recognize wild=type (WT) CNAα in its unphosphorylated form, but that it does recognize phosphomimetic antibody mutants S469E and S462E/S469E. Total CNAα levels are assessed using a phosphorylation nonspecific anti-CNAα antibody (bottom lanes).

### DETAILED DESCRIPTION

This disclosure concerns the discovery that phosphorylation at position S469 of the protein encoded by Ppp3ca gene, the alpha paralog of the catalytic subunit of calcineurin (CNAα), may serve as a marker for neuronal activity. Specific polyclonal antibodies recognizing phospho-S469 CNAα were found to enable sensitive measurement of changes in neuronal activity. Such phosphospecific antibodies may allow for relatively rapid measurement of neuronal activity, for instance in various diagnostic and screening assays of diseases and therapeutics.

### Definitions

The titles, headings and subheadings provided herein should not be interpreted as limiting the various aspects of the disclosure. Accordingly, the terms defined immediately below are more fully defined by reference to the specification in its entirety.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Systeme International de Unites (SI) accepted form. Numeric ranges are inclusive of the numbers defining the range. Measured values are understood to be approximate, taking into account significant digits and the error associated with the measurement.

As utilized in accordance with the present disclosure, the following terms, unless otherwise indicated, shall be understood to have the following meanings:

As used herein, “calcineurin A alpha” or “calcineurin alpha” or “CNAα” refers herein to the protein encoded by the Ppp3ca gene, also known as the alpha paralog of the catalytic subunit of calcineurin. CNAα may also be referred to as the serine/threonine-protein phosphatase 2B catalytic subunit alpha isoform, the CAM-PRP catalytic subunit, or the calmodulin-dependent calcineurin A subunit alpha isoform. CNAα is a calcium-dependent, calmodulin-stimulated serine/threonine protein phosphatase. An exemplary sequence of CNAα is presented as SEQ ID NO: 3 (UniProt Q08209). An exemplary sequence of CNAα with removal of the initiator methionine is amino acids 2-521 of SEQ ID NO: 3. Unless specifically noted otherwise, the term CNAα herein refers to human CNAα.

As used herein, “phospho-S469 CNAα” refers herein to CNAα that is phosphorylated at position S469. The phosphorylation of CNAα at position S469 may be neuronal activity-dependent.

The term “isolated” refers to a molecule that has been separated from at least some of the components with which it is typically found in nature or with which it has been produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide.

The term “antibody” as used herein refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term antibody includes, but is not limited to, fragments that are capable of binding antigen (“antigen binding fragments”), such as, but not limited to, Fv, single-chain Fv (scFv), Fab, Fab′, and (Fab′)2. The term antibody also includes, but is not limited to, chimeric antibodies, humanized antibodies, and antibodies of various species such as mouse, human, cynomolgus monkey, etc. Antibodies herein may also be polyclonal or monoclonal antibodies. They may also be multispecific or bispecific, and they may also be conjugated to other molecules, such as beads or other detection reagents.

In certain embodiments, an antibody provided herein is a multispecific antibody. As used herein, “multispecific antibodies” are monoclonal antibodies that have binding specificities for at least two different sites. In some embodiments, an antibody provided herein is a “bispecific antibody.” Bispecific antibodies can be prepared as full-length antibodies or antibody fragments. In some embodiments, the antibody is a diabody, triabody, or tetrabody. Diabodies are antibody fragments with two antigen-binding sites that may be bivalent or bispecific.

As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some such embodiments, the heavy chain is the region of the antibody that comprises the three heavy chain CDRs and the light chain in the region of the antibody that comprises the three light chain CDRs.

As used herein, “phospho-S469 CNAα antibody” refers herein to an antibody specific affinity to phospho-S469 CNAα.

Antibodies that “bind specifically to” or have “specific affinity for” or that “specifically recognize” a particular target such as phospho-S469 CNAα, or similar terms, refer to antibodies that bind to the target with high enough affinity and specificity such that the antibodies show little to no binding to other, possibly competing targets in assays in which they are used. Exemplary competing targets could include any of unphosphorylated CNAα or other calcineurin A paralogs such as CNAβ and CNAγ.

The term “heavy chain variable region” refers to a region comprising heavy chain HVR1, framework (FR) 2, HVR2, FR3, and HVR3. In some embodiments, a heavy chain variable region also comprises at least a portion of an FR1 and/or at least a portion of an FR4.

The term “heavy chain constant region” refers to a region comprising at least three heavy chain constant domains, C_{H}1, C_{H}2, and C_{H}3. Nonlimiting exemplary heavy chain constant regions include γ, δ, and α. Nonlimiting exemplary heavy chain constant regions also include ϵ and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an c constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “light chain variable region” refers to a region comprising light chain HVR1, framework (FR) 2, HVR2, FR3, and HVR3. In some embodiments, a light chain variable region also comprises an FR1 and/or an FR4.

The term “light chain constant region” refers to a region comprising a light chain constant domain, C_{L}. Nonlimiting exemplary light chain constant regions include λ and κ.

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence.

The term “hypervariable region” or “HVR” refers to each of the regions of an antibody variable domain which are hypervariable in sequence and/or form structurally defined loops (“hypervariable loops”). Generally, native four-chain antibodies comprise six HVRs; three in the V_{H} (H1, H2, H3), and three in the V_{L} (L1, L2, L3). HVRs generally comprise amino acid residues from the hypervariable loops and/or from the “complementarity determining regions” or “CDRs”, the latter being of highest sequence variability and/or involved in antigen recognition. The terms hypervariable regions (HVRs) and complementarity determining regions (CDRs), are used herein interchangeably in reference to portions of the variable region that form the antigen binding regions.

A “chimeric antibody” as used herein refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, a chimeric antibody comprises at least one rat variable region and at least one mouse constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is a Fab, an scFv, a (Fab′)2, etc.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

Human antibodies can be made by any suitable method. Nonlimiting exemplary methods include making human antibodies in transgenic mice that comprise human immunoglobulin loci. See, e.g., Jakobovits et al., Proc. Natl. Acad. Sci. USA 90: 2551-55 (1993); Jakobovits et al., Nature 362: 255-8 (1993); Lonberg et al., Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806. Nonlimiting exemplary methods also include making human antibodies using phage display libraries. See, e.g., Hoogenboom et al., J. Mol. Biol. 227: 381-8 (1992); Marks et al., J. Mol. Biol. 222: 581-97 (1991); and PCT Publication No. WO 99/10494.

As used herein, “phosphospecific antibody” refers herein to an antibody that has greater affinity for an epitope when the epitope comprises a phosphorylated amino acid at a specific position compared to the same epitope with an unphosphorylated amino acid at the same position. A phosphospecific antibody may be utilized to determine the state of phosphorylation of a specific residue under different conditions.

As used herein, “induced pluripotent stem cells” or “IPSCs” refers herein to any stem cells that are derived from differentiated cells. These differentiated cells may be non-fetal or adult cells. IPSCs may be non-fetal or adult cells that are reprogrammed into stem cells. These reprogrammed stem cells may be pluripotent stem cells. IPSCs may be a source of patient-derived stem cells, wherein the stem cells comprise the genetic makeup of the patient. IPSCs may be used for personalized drug discovery based on their makeup unique to the patient of interest.

A “chemical compound” as used herein that has or may have an impact on neuronal activation or neuronal activity in a sample. This term broadly encompasses not only small molecule chemicals, but also macromolecules such as polypeptides.

As used herein, “neuronal activation” refers herein to any cellular change in a neuron from its resting state to an excited state. Examples of a cellular change associated with neuronal activity may be a change in an intracellular process, synaptic transmission, release of growth or neurotrophic factors, or dendritic or synaptic growth. Thus, neuronal activity may comprise numerous properties of neurons such as excitability of individual neurons, communication between neurons, and synaptogenesis.

As used herein, the term “neuronal activity” or “neuronal activity level” refers to the extent to which a neuron is activated, i.e., the degree of activation of a neuron. When applied to a sample comprising multiple neurons, neuronal activity or neuronal activity level refers to the extent to which neurons in the sample are activated, i.e., the degree of activation of neurons in the sample. As disclosed herein, neuronal activation and neuronal activity level may be measured by using markers such as changes in gene expression or phosphorylation of CNAα.

As used herein, “long term potentiation” or “LTP” refers to a persistent strengthening of synaptic transmission based on a recent pattern of neuronal activity. In some embodiments, LTP is a long-lasting increase in transmission between two neurons. LTP may be induced by high-frequency stimulation of neurons, such as by electrical stimulation of a brain slice. A “chemical LTP” or “cLTP” protocol refers to LTP that is produced by treatment of neurons with one or more chemical reagents. A cLTP protocol may be used with neurons in culture via treatment with agents that promote synaptic activity.

As used herein, “long term depression” or “LTD” refers to a persistent weakening of synaptic transmission based on a recent pattern of neuronal activity. In some embodiments, LTD is a long-lasting decrease in transmission between two neurons.

As used herein, the term “synaptic plasticity” or “neural plasticity” refers to the ability of synaptic transmissions to strengthen or weaken over time in response to increases or decreases in their activity.

The term “neurological disorder” or “neurological disease” are used interchangeably herein to refer to a disorder of the nervous system (e.g., brain, spinal cord and nerves). There are more than 600 disorders of the nervous system, including disorders that psychiatric and neurodevelopmental in nature. Many neurological disorders may be associated with changes in neuronal activation and can be studied using neural tissue slices, for example from animal models, or using cultured neurons from animals or humans, such as iPSCs from human subjects or mammalian hippocampal, cortical, striatum, cerebellar granule, or other mammalian neuronal cells. Examples of neurological disorders that may impact activation of neurons, include, but are not limited to, Alzheimer's disease, alcoholic Korsakoff disease (KS), multiple sclerosis, Parkinson's disease, Down's syndrome, Williams syndrome, Specific Language Impairment, and Attention Deficit Hyperactivity Disorder (ADHD), spinal cord injury, stroke, traumatic brain injury, hearing loss, neurodegenerative diseases such as amyotrophic lateral sclerosis (ALS), Huntington's disease, epilepsy, autism, fragile X syndrome, and cerebral ischemia as well as alcoholism, alcohol withdrawal, benzodiazepine withdrawal, and hypoglycemia.

The terms “subject” and “patient” are used interchangeably herein to refer to a mammal that would be benefitted by the compositions and/or methods of the invention. In some embodiments, the mammal is a human. In some embodiments, the mammal is a non-human species, including, but not limited to, rodents, simians, felines, canines, equines, bovines, porcines, ovines, caprines, mammalian laboratory animals, mammalian farm animals, mammalian sport animals, and mammalian pets.

As used herein, the term “detection reagent” refers to one or more reagents for detecting the level of phospho-S469 CNAα antibodies in the biological sample. In some embodiments, the detection reagent is at least one of a bead or similar substrate, a fluorescent label, a chemiluminescent label, a secondary antibody, and a tertiary antibody. In some embodiments, the antibody may be conjugated to one or more detection reagents while in other embodiments, the antibody and detection reagents may be separate or on separate molecules.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

### CNAα and Exemplary Phospho-S469 CNAα Antibodies

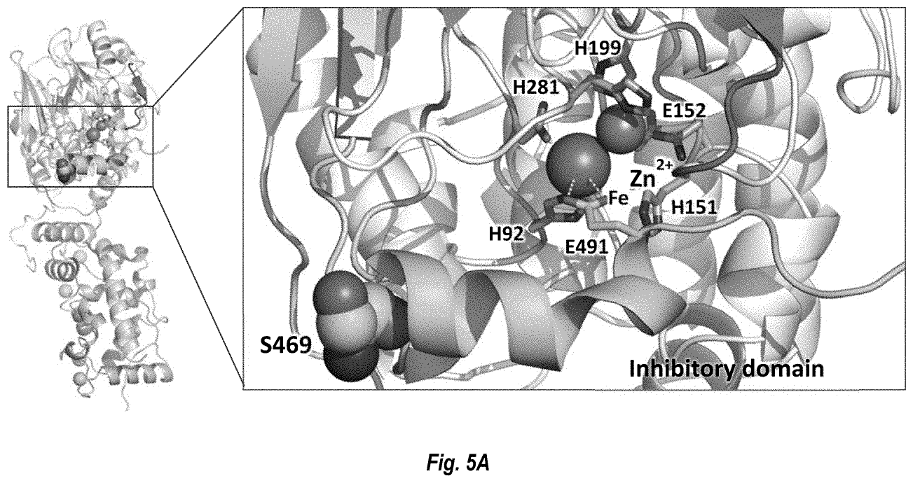

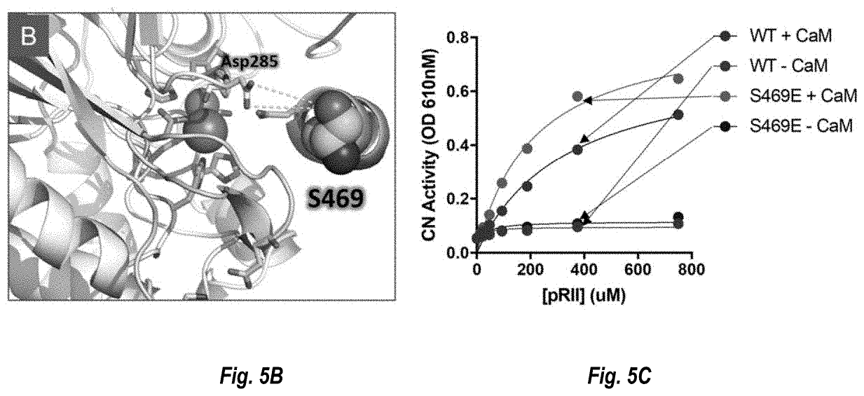

The calcineurin holoenzyme is comprised of a ˜56 kD catalytic subunit, calcineurin A (CNA) and a ˜25 kD regulatory subunit, calcineurin B (CNB). There are three paralogs of the catalytic subunit, PPP3CA, PPP3CB, and PPP3CC, which encode the calcineurin A α, β, and y isoforms, respectively. The three paralogs have different amino acid sequences near position 469, as noted in SEQ ID NOs: 1, 5, and 6 depicted herein. The catalytic subunit is comprised of four domains, a catalytic domain, a regulatory subunit binding domain, a calmodulin (CaM) binding domain, and the autoinhibitory domain (AID). As seen in FIG. 5A, the AID wraps to sterically block the catalytic site of the enzyme, and the binding of active CaM reduces that autoinhibition, substantially enhancing phosphatase activity. Serine 469 of CNAα resides at the N-terminal end of the AID. Thus, its phosphorylation might alter enzyme activity by modulating the binding of the AID to the catalytic site.

As described above, phospho-S469 CNAα antibodies are antibodies that specifically recognize the phosphorylated S469 form of CNAα. Thus, for example, such antibodies may distinguish phosphorylated from unphosphorylated CNAα in assays described herein. In some embodiments, therefore, the antibodies may specifically bind to a phosphorylated CNAα epitope including the S469 position but may not specifically bind to the corresponding unphosphorylated epitope or to an epitope from a mutant CNAα protein in which the serine (S) at position 469 is substituted with another amino acid such as glutamate (E). In some embodiments, the antibodies do not specifically recognize either CNAβ or CNAγ. In some embodiments, the antibodies do not specifically recognize CNAα domains other than the autoinhibitory domain (AID).

In some embodiments, the antibodies may specifically recognize an epitope comprising the amino acid sequence of SEQ ID NO:1. In some embodiments, the antibodies may specifically recognize an epitope consisting of the amino acid sequence of SEQ ID NO:1. In some embodiments, the antibodies may specifically recognize the amino acid in an epitope comprising the amino acid sequence of SEQ ID NO:1 but may not specifically recognize an epitope that (a) comprises the amino acid sequence of SEQ ID NO:2 and does not comprise the amino acid sequence of SEQ ID NO:1. In some embodiments, the antibodies may specifically recognize the amino acid in an epitope comprising the amino acid sequence of SEQ ID NO:1 but may not specifically recognize an epitope consisting of amino acid sequence of SEQ ID NO:2.

In some embodiments, phospho-S469 CNAα antibodies may be obtained, for example, by selecting for antibodies that specifically recognize the amino acid an epitope comprising the amino acid sequence of SEQ ID NO:1, or consisting of the amino acid sequence of SEQ ID NO:1. In some embodiments, the epitope comprising the amino acid sequence of SEQ ID NO:1 comprises no more than 10, 9, 8, 7, 6, 5, 4, 3, 2 or 1 amino acids upstream of the N-terminus of SEQ ID NO:1. In some embodiments, the epitope comprising the amino acid sequence of SEQ ID NO:1 comprises no more than 10, 9, 8, 7, 6, 5, 4, 3, 2 or 1 amino acids downstream of the C-terminus of SEQ ID NO:1. In some embodiments, the epitope comprising the amino acid sequence of SEQ ID NO:1 comprises no more than 10, 9, 8, 7, 6, 5, 4, 3, 2 or 1 amino acids upstream of the N-terminus of SEQ ID NO:1 and comprises no more than 10, 9, 8, 7, 6, 5, 4, 3, 2 or 1 amino acids downstream of the C-terminus of SEQ ID NO:1. In some embodiments, the epitope comprising the amino acid sequence of SEQ ID NO:1 comprises no more than 10, 9, 8, 7, 6, 5, 4, 3, 2 or 1 amino acids upstream of the N-terminus of SEQ ID NO:1 and comprises no more than 5, 4, 3, 2 or 1 amino acids downstream of the C-terminus of SEQ ID NO:1. In some embodiments, the epitope comprising SEQ ID NO:1 does not overlap in sequence with SEQ ID NO:2, which is further downstream toward the C terminus of the CNAα protein.

In some embodiments, antibodies are further screened to ensure that they do not specifically bind to the unphosphorylated CNAα protein or to CNAβ or CNAγ. This may be done, for instance, by selecting for antibodies that do not specifically bind to nearby epitopes on the C-terminal portion of the CNAα protein, such as SEQ ID NO:2, or by selecting for antibodies that do not bind specifically to portions of the CNAα protein outside of the AID, or by selecting for antibodies that do not specifically bind to the unphosphorylated form of SEQ ID NO:1 provided in SEQ ID NO:4, or to the equivalent portions of the CNAβ and CNAγ proteins provided in SEQ ID NOs:5 and 6 herein. For example, the portion of SEQ ID NO:1 N-terminal to S469 has a different amino acid sequence in the CNAβ and CNAγ paralogs of calcineurin A, such that an antibody that specifically recognizes SEQ ID NO:1 may not specifically bind to either CNAβ or CNAγ.

In some embodiments, the phospho-S469 CNAα antibodies may bind specifically not only to the phosphorylated human protein, but also to the phosphorylated murine, rat, or cynomolgus protein, for example, as the SEQ ID NO:1 amino acid sequence from the human CNAα AID is 100% conserved between the human, murine, and rat CNAα proteins.

The phospho-S469 CNAα antibodies herein may comprise a heavy chain and a light chain, wherein the heavy chain comprises a heavy chain complementary determining region 1 (HCDR1) comprising the amino acid sequence of SEQ ID NO: 11; a HCDR2 comprising the amino acid sequence of SEQ ID NO: 12; and a HCDR3 comprising the amino acid sequence of SEQ ID NO: 13; and wherein the light chain comprises a light chain complementary determining region 1 (LCDR1) comprising the amino acid sequence of SEQ ID NO: 14; a LCDR2 comprising the amino acid sequence of SEQ ID NO: 15; and a LCDR3 comprising the amino acid sequence of SEQ ID NO: 16. In some embodiments, the heavy chain of the antibody comprises a heavy chain variable region with an amino acid sequence that is at least 90%, at least 95%, at least 97%, at least 98%, or at least 99% identical to the amino acid sequence of SEQ ID NO: 7. In some embodiments the light chain of the antibody comprises a light chain variable region amino acid sequence that is at least 90%, at least 95%, at least 97%, at least 98%, or at least 99% identical to the amino acid sequence of SEQ ID NO: 9. In some embodiments, the heavy chain variable region of the antibody comprises the amino acid sequence of SEQ ID NO: 7. In some embodiments, the light chain variable region of the antibody comprises the amino acid sequence of SEQ ID NO: 9. In some embodiments, the heavy chain variable region of the antibody comprises the amino acid sequence of SEQ ID NO: 7 and the light chain variable region of the antibody comprises the amino acid sequence of SEQ ID NO: 9.

Phospho-S469 CNAα antibodies herein may include, but are not limited to, antigen binding fragments, humanized antibodies, chimeric antibodies, mouse or rat antibodies, cynomolgus antibodies, and human antibodies. The antibodies herein may also be conjugated to various molecules including chemical labels and beads or other substrates used in detection systems.

### Chimeric and Humanized Antibodies

In some embodiments, the antibodies are chimeric or humanized antibodies. In some embodiments, a phospho-S469 CNAα antibody is a chimeric antibody. Exemplary chimeric antibodies may comprise one or both of a heavy chain variable region and a light chain variable region of a phospho-S469 CNAα antibody, such as a murine antibody, grafted onto human constant regions. In some embodiments, a phospho-S469 CNAα antibody comprises at least one non-human variable region and at least one human constant region. In some such embodiments, all of the variable regions of a phospho-S469 CNAα antibody are non-human variable regions, and all of the constant regions of a phospho-S469 CNAα antibody are human constant regions. In some embodiments, one or more variable regions of a chimeric antibody are mouse variable regions. The human constant region of a chimeric antibody need not be of the same isotype as the non-human constant region, if any, that it replaces. Chimeric antibodies are discussed, e.g., in U.S. Pat. No. 4,816,567; and Morrison et al. Proc. Natl. Acad. Sci. USA 81: 6851-55 (1984).

As noted above, a humanized antibody is an antibody in which at least one amino acid in a framework region of a non-human variable region has been replaced with the amino acid from the corresponding location in a human framework region. A humanized antibody may also comprise human constant regions. In some embodiments, at least two, at least three, at least four, at least five, at least six, at least seven, at least eight, at least nine, at least 10, at least 11, at least 12, at least 15, or at least 20 amino acids in the framework regions of a non-human variable region are replaced with an amino acid from one or more corresponding locations in one or more human framework regions.

In some embodiments, some of the corresponding human amino acids used for substitution are from the framework regions of different human immunoglobulin genes. That is, in some such embodiments, one or more of the non-human amino acids may be replaced with corresponding amino acids from a human framework region of a first human antibody or encoded by a first human immunoglobulin gene, one or more of the non-human amino acids may be replaced with corresponding amino acids from a human framework region of a second human antibody or encoded by a second human immunoglobulin gene, one or more of the non-human amino acids may be replaced with corresponding amino acids from a human framework region of a third human antibody or encoded by a third human immunoglobulin gene, etc. Further, in some embodiments, all of the corresponding human amino acids being used for substitution in a single framework region, for example, FR2, need not be from the same human framework. In some embodiments, however, all of the corresponding human amino acids being used for substitution are from the same human antibody or encoded by the same human immunoglobulin gene.

In some embodiments, an antibody is humanized by replacing one or more entire framework regions with corresponding human framework regions. In some embodiments, a human framework region is selected that has the highest level of homology to the non-human framework region being replaced. In some embodiments, a humanized antibody is a CDR-grafted antibody, in which CDRs from a non-human antibody are grafted into a set of human framework regions and optionally also human constant regions.

In some embodiments, following CDR-grafting, one or more framework amino acids are changed back to the corresponding amino acid in the non-human framework region, e.g. a mouse framework region. Such “back mutations” are made, in some embodiments, to retain one or more mouse framework amino acids that appear to contribute to the structure of one or more of the CDRs and/or that may be involved in antigen contacts and/or appear to be involved in the overall structural integrity of the antibody. In some embodiments, ten or fewer, nine or fewer, eight or fewer, seven or fewer, six or fewer, five or fewer, four or fewer, three or fewer, two or fewer, one, or zero back mutations are made to the framework regions of an antibody following CDR grafting.

In some embodiments, a humanized antibody described herein comprises one or more human constant regions. In some embodiments, the human heavy chain constant region is of an isotype selected from IgA, IgG, and IgD. In some embodiments, the human light chain constant region is of an isotype selected from κ and λ. In some embodiments, a chimeric or humanized antibody described herein comprises a human IgG constant region. In some embodiments, a humanized antibody described herein comprises a human IgG1, IgG2, IgG3, or IgG4 heavy chain constant region.

An antibody may be humanized by any method. Nonlimiting exemplary methods of humanization include methods described, e.g., in U.S. Pat. Nos. 5,530,101; 5,585,089; 5,693,761; 5,693,762; 6,180,370; Jones et al., Nature 321: 522-525 (1986); Riechmann et al., Nature 332: 323-27 (1988); Verhoeyen et al., Science 239: 1534-36 (1988); and U.S. Publication No. US 2009/0136500.

Further exemplary chimeric or humanized antibodies are bivalent (i.e., having two heavy chains and two light chains) antibody versions of antibodies disclosed herein.

### Exemplary Human Antibodies

Human antibodies can be made by any suitable method. Nonlimiting exemplary methods include making human antibodies in transgenic mice that comprise human immunoglobulin loci. See, e.g., Jakobovits et al., Proc. Natl. Acad. Sci. USA 90: 2551-55 (1993); Jakobovits et al., Nature 362: 255-8 (1993); Lonberg et al., Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806.

Nonlimiting exemplary methods also include making human antibodies using phage display libraries. See, e.g., Hoogenboom et al., J. Mol. Biol. 227: 381-8 (1992); Marks et al., J. Mol. Biol. 222: 581-97 (1991); and PCT Publication No. WO 99/10494.

In some embodiments, a human phospho-S469 CNAα antibody comprises one or more human constant regions. In some embodiments, the human heavy chain constant region is of an isotype selected from IgA, IgG, and IgD. In some embodiments, the human light chain constant region is of an isotype selected from κ and λ. In some embodiments, a human antibody described herein comprises a human IgG constant region. In some embodiments, a human antibody described herein comprises a human IgG1, IgG2, IgG3, or IgG4 heavy chain constant region.

### Assays Using Phospho-S469 CNAα Antibodies

The antibodies herein have a number of uses in assays of neural tissue or cultured neurons. For example, neuronal activity and long term potentiation (LTP) of neurons may be measured and analyzed in tissue samples or in neuronal culture samples. In such samples, neuronal activation and neuronal activity level are commonly measured by tracking expression of immediate early genes (IEGs) such as c-fos, zif268/epr-1, or arc/arg3. 1 following exposure of neurons to a stimulus, thus linking neuronal activity to changes in gene expression. Expression may be tracked at the protein level, such as by immunohistochemistry (IHC) using antibodies to the gene products, or at the RNA level, such as by in situ hybridization, e.g. catFISH assays (cellular compartment analysis of temporal activity by fluorescence in situ hybridization). (See, e.g., M. Sheng & M. E. Greenberg, Neuron 4: 477-485 (1990); J. F. Guzowski, Nature Neurosci. 2: 1120-24 (1999), A. V. Tzingounis & R. A. Nicoll, Neuron 52: 403-7 (2006); R. Chandra & M. K. Lobo, Frontiers in Behavioral Neurosci. 11(Article 112) (June, 2017).) These assays, however, do not provide a real-time analysis of neuronal activation as detectable gene expression may not begin until, for example, 20-45 minutes after induction with a stimulant and may continue for several hours after stimulation. Thus, these assays may record changes in neuronal activity on a time-scale of, for example, 30 minutes, or an hour, or several hours, making them slow to perform and limiting their ability to detect transient or weak responses.

In contrast, the experiments herein show that phosphorylation of CNAα at S469 occurs on a time-scale of a few minutes following exposure of neurons to stimulants. Furthermore, phosphorylation of S469 is highly sensitive to changes in neuronal activity level in cultured neurons and brain slices, meaning that an assay based on tracking that phosphorylation may have a broad dynamic range and may be highly sensitive to small changes in neuronal activity level. Accordingly, tracking phosphorylation of S469 may be used to measure levels of neuronal activity in a culture or brain slice immediately prior to fixation or lysis of the material, and generally on a time-scale of a few minutes. In some embodiments, tracking phosphorylation of S469 using phospho-S469 CNAα antibodies may be a significantly faster neuronal activity assay than those based on changes in gene expression. Thus, in some embodiments, changes in neuronal activity may be measured on a time-scale of, for example, 1-20 minutes, 1-10 minutes, or 1-5 minutes. For example, in some embodiments, the level of antibody retained by a sample of neurons may be recorded simultaneously with addition of a potential stimulant or inhibitor of neuronal activity, or 1-20 minutes, 1-10 minutes, or 1-5 minutes after that addition. Furthermore, data herein show that phospho-S469 CNAα antibodies can be used to mark neuronal activity levels in neuronal cultures as well as in fixed or processed brain tissue samples.

Assays using phospho-S469 CNAα as a proxy for neuronal activation may, for example, be used to study the impact of genetic alterations on the activity of cultured neurons or neurons derived from tissue samples taken from human or mammalian patients. In some embodiments, the neurons are derived from IPS cells generated from the tissue samples from human or mammalian patients. They may also be used to screen potential drug compounds that might enhance or depress neuronal activity. They may be used to develop treatment strategies for individual patients or classes of patients by determining whether available therapeutic agents have an impact on a patient's neuronal activation, thus helping to determine what therapeutic agents a patient will respond to. They may be used to test therapeutic agents that are not intended to impact neuronal activity to ensure that the agents have no effect.

In some embodiments, the methods comprise measuring neuronal activity in a biological sample, such as a tissue sample comprising neurons or a sample comprising cultured neurons, by using the amount or concentration of phospho-S469 CNAα in the sample as a marker for neuronal activity. In some embodiments, the methods comprise using a signal obtained from the specific binding of phospho-S469 CNAα antibodies to phospho-S469 CNAα as a marker for neuronal activity. In some embodiments, the methods measure neuronal activity following exposure of the sample to one or more chemical or physical changes, such as exposure to one or more chemical compounds (e.g. neurotransmitters or potential therapeutic agents) or to one or more physical signals (e.g. electromagnetic waves) that may have an impact, either positive or negative, on neuronal activity level. In some embodiments, the methods comprise obtaining a sample comprising neurons, such as a tissue sample comprising neurons or a sample comprising cultured neurons, exposing the sample to one or more chemical compounds or physical signals and then also exposing the sample to the phospho-S469 CNAα antibodies and detecting the level of the phospho-S469 CNAα antibodies retained by the sample, whereby the level of phospho-S469 CNAα antibodies retained by the sample provides a marker to indicate the neuronal activity level in the sample. In some embodiments, the amount of S469 phosphorylation can be measured within 1-20 minutes, 1-10 minutes, 3-8 minutes, 3, 4, 5, 6, 7, or 8 minutes of exposure to the chemical compounds or physical signals impacting neuronal activity. Such assays may be performed using a variety of different antibody detection formats, as described below, for example, using fluorescent or chemiluminescent stains to identify the extent of phospho-S469 CNAα antibodies retained by the sample. Optionally contrasting stains of different colors or wavelengths may be used to visualize neurons in the sample. In some embodiments, after exposing the sample to the phospho-S469 CNAα antibodies, one or more wash steps is performed to remove unbound antibodies prior to detecting the level of antibodies retained by the sample.

In some embodiments, such an assay may be used as a screen for potential therapeutic compounds. For example, in some embodiments, the one or more chemical compounds assayed in the assay can be potential therapeutics, in order to assess whether they improve neuronal activity. For instance, such a therapeutic screening assay could be run using a biological sample acting as a model for diseased neurons, for instance a brain tissue sample or other tissue sample from an animal model of a neurological disease or a human cultured neuron sample from a patient with a neurological disease or a with a related genetic mutation.

In some embodiments, for instance using cultured neuron cells from a patient with a neurological disease, the methods herein can be used to determine which therapeutics the patient's neurons may be responsive to and which therapeutics the patient's neurons may not be responsive to. For example, after screening a patient's neurons to several potential therapeutics, an appropriate treatment for the patient may be determined and administered to the patient. Accordingly, the methods herein also comprise methods of treating a patient with a neurological disorder by first obtaining the results of such a therapeutic screen on a sample derived from the patient, such as a cultured neuron sample, and then from those results, administering at least one therapeutic agent identified in the screen to increase the activity of neurons in the sample. In some embodiments, the methods comprise not administering to the patient any therapeutic agent determined from the methods not to increase the activity of the neurons in the sample. In some embodiments, the methods herein comprise methods of treating a patient with a neurological disorder by first obtaining the results of such a therapeutic screen on a sample obtained from the patient or derived from the patient's cells, such as a cultured neuron sample, and then from those results, administering at least one therapeutic agent identified in the screen to decrease the activity of neurons in the sample. In some embodiments, the at least one therapeutic agent identified in the screen to decrease the neuronal activity is administered to a patient with conditions including, but not limited to, Many neurological disorders may be associated with changes in neuronal activation and can be studied using neural tissue slices, for example from animal models, or using cultured neurons from animals or humans, such as iPSCs from human subjects or mammalian hippocampal, cortical, striatum, cerebellar granule, or other mammalian neuronal cells.

In some embodiments, the desired modulation of neuronal activity, an increase or decrease, may depend on the genetic variation and brain circuit being analyzed.

For example, diseases in which such screens may be useful include neurological disorders that are believed to impact neuronal activity, including long-term potentiation and synaptic plasticity.

In some embodiments, neural tissue samples, for example from animal models, or neuronal cultures utilized in the assays may be associated with neurological disorders such as schizophrenia, bipolar disorder, epilepsy; neurological diseases, disorders or conditions including, but not limited to, Alzheimer's disease, alcoholic Korsakoff disease (KS), multiple sclerosis, Parkinson's disease, Down's syndrome, Williams syndrome, Specific Language Impairment, and Attention Deficit Hyperactivity Disorder (ADHD). Neurological disorders that may have an impact on the activation of neurons, and thus for which tissue samples from a model system or neuronal cultures can be subjected to therapeutic screening assays herein include spinal cord injury, stroke, traumatic brain injury, hearing loss, neurodegenerative diseases such as amyotrophic lateral sclerosis (ALS), Huntington's disease, epilepsy, autism, fragile X syndrome, and cerebral ischemia as well as conditions such as alcoholism and alcohol withdrawal, benzodiazepine withdrawal, and hypoglycemia.

The methods herein may also be used to screen potential therapeutic agents to ensure that agents that are not intended to impact neuronal activity, in fact, do not impact such activity. For example, such screens could be performed in mammalian tissue samples or cultured neuron samples as a type of toxicology screen for potential therapeutics.

In some embodiments, the methods herein may be used in the study of patients with neurological disease, for example patients who display genetic alterations. Accordingly, the methods herein can be used to determine and compare neuronal activity in cultured neurons or tissue samples from subjects with and without a particular genetic mutation in order to determine the impact of the mutation on neuronal activity. Such screens may also be used to determine which therapeutics may be most effective in treating patients having a particular genetic mutation.

In any of the methods herein, the methods may be conducted to assay the chemical long-term potentiation (cLTP) of neurons in the sample after exposure to one or more chemical compounds, for example, by first activating neurons in the sample with a chemical compound, and then attempting to re-activate neurons in the sample with at least one compound to be tested.

Assays herein may be run in a high-throughput format, for example, by splitting the sample, such as a cultured neuron sample, into multiple sections, such as in a 32, 48, or 96 well plate, where each portion of the plate is exposed to a different chemical compound.

### Exemplary Assay Formats

### LTP, LTD, and Synaptic Plasticity Assays

Neuronal activity can be used to measure the processes of synaptic plasticity, including LTP and LTD. These processes are considered to be cellular correlates of learning and memory that are dependent on molecular and cellular changes in neurons (see Luscher C and Malenka R C, Cold Spring Harb Perspect Biol 4:a005710 (2012)). LTP and LTD have been characterized by mechanisms of gene expression (occurring within hours) and maintenance (occurring over the course of days). To measure LTP or LTD, neurons in a sample may first be activated by a stimulus, then re-activated by the same or a different stimulus. After an LTP protocol, stimulation of a presynaptic neuron will produce a larger response in the post-synaptic neuron than in the original stimulation. Conversely, after an LTD protocol, stimulation of a presynaptic neuron will produce a smaller response in the post-synaptic neuron than in the original stimulation. LTP and LTD are often studied in slices of brain tissue, and chemical LTP may be studied, for example, in cultured neurons.

Specific patterns of activity may induce both LTP and LTD. For example, the initial step of LTP may be a presynaptic release of neurotransmitter and activation of ionotropic glutamate receptors (such as AMPA and NMDA receptors) that cause a depolarization of the postsynaptic neuron, which activates intracellular signaling cascades that lead to altered synaptic efficacy. In contrast, LTD is associated with repeated activation of presynaptic neurons at low frequencies without postsynaptic activity.

LTP and LTD can be time-consuming to study with current methods. In each case, intracellular changes in neurons that occur over a short-term may be important first-steps in these processes. As such, assays that measure neuronal activity on a short time scale would be valuable predictors of the cellular changes underlying LTP, LTD, and synaptic plasticity.

LTP and LTD may be measured in assays in which neurons in a sample are first stimulated with a chemical or physical signal. Then, neurons are exposed a second tome to the same signal or to new test signal, such as, for example, from a potential therapeutic compound, to determine how this second signal impacts neuronal activity. In some embodiments, for example, LTP and LTD can be measured using a multielectrode array (MEA), such as described in MEA Application Note: Acute Hippocampus Slices from Rat or Mouse; Preparation, Recording, and Data Analysis. Multi Channel Systems (2017). In such an assay, oxygenated artificial cerebrospinal fluid is prepared in a slice storage chamber, and hippocampal slices are prepared using a vibratome. The slice is placed on the MEA, and baseline measurements are recorded. Then stimulation electrodes are used to excite pathways in the slice and induce LTP. Exemplary stimulation protocols for inducing LTP include one hundred pulses at 100 Hz for one second (which may be repeated at regular intervals). Following the LTP induction, changes in response to the same stimulation are compared to the baseline measurements. Exemplary measures to monitor changes in synaptic transmission by LTP include changes in slope of the excitatory postsynaptic potential (EPSP) or changes in the EPSP amplitude.

In some embodiments, the level of phospho-S469 CNAα is used as a marker for measuring LTP or LTD. In some such embodiments, neurons in a sample are first stimulated with a chemical or physical sample. Then neurons are stimulated again with the same or a different signal. Levels of phospho-S469 CNAα after such an LTP induction protocol may be used to measure changes in neuronal activity in response to further stimulation of the sample.

In some embodiments, LTP may also be measured by stimulation and monitoring of EPSPs without use of MEA. For example, LTP may be induced at synapses between CA3 and CA1 pyramidal neurons by tetanic stimulation of Schaffer collaterals (see Luscher and Malenka 2012). For example, LTP protocols may comprise delivery of signals such as high-frequency stimulations of one or more trains of pulses at 50-100 Hz for 1 second. In contrast, LTD protocols may comprise delivery of low-frequency stimulations at 1-3 Hz for 5-15 minutes. In some embodiments, the levels of phospho-S469 CNAα after an LTP induction protocol induced by one or more trains of pulses in a sample, such as a brain slice sample, may be used to measure changes in neuronal activity in response to high-frequency stimulation of a sample. In some embodiments, the levels of phospho-S469 CNAα after an LTD induction protocol induced by low-frequency pulses in a sample, such as a brain slice sample, may be used to measure changes in neuronal activity in response to low-frequency stimulation of a sample.

In some embodiments, LTP may be induced in cultured neurons, for example, using chemical stimulation signals. In some embodiments, LTP may be induced via treatment of cultured neurons with agents known to increase synaptic activity. Cultures may be treated with one or more agents such as bicuculline (an antagonist of GABA_{A} receptors), strychnine (an antagonist of glycine and acetylcholine receptors), and glycine (a coagonist of NMDA glutamatergic receptors), for example. In some embodiments, the levels of phospho-S469 CNAα after a LTP protocol with cultured neurons may be used to measure changes in neuronal activity in response to agents that stimulate synaptic transmission.

### iPSC-Derived Neuronal Culture Assays

In some embodiments, a sample to be assayed in methods herein may be a neural culture sample. In some embodiments, mammalian neural cultures may be used as models for diseases or particular genetic conditions. In addition, recent work with induced pluripotent stem cell (IPSC)-derived neurons has provided the opportunity to develop in vitro diagnostic assays using neurons with the genetic make-up of patients without the need for retrieving neuronal tissue (see Dolmetsch R and Geschwind D H, Cell 145(6): 831-834 (2011) and Zhang Y et al., Stem Cell Reports 8:648-658 (2017)). For example, diagnostic assays assessing activity of IPSC-derived neurons from patients could be used to determine the impact of specific genetic mutations or variations, which may be seen in patients with neurologic diseases (e.g., in patients with epilepsy). Thus, in some embodiments, a neural culture sample used in assays herein may comprise IPSCs.

IPSC-derived neurons from subjects in culture could be used in an in vitro assay measuring neuronal activity to test the ability of proposed therapeutic agents to alter neuronal activity in specific patient populations, for example. In vitro assays using IPSC-derived neurons to measure neuronal activity using phospho-S469 CNAα as a marker of that activity could thus be used in drug development of patient-specific, personalized therapies for subjects with specific genetic mutations or variations.

### Antibody Detection Platforms

A number of different assay formats can be developed to detect phosphospecific antibodies in an in vitro screening assay. High-throughput visualization systems may be used to assess changes in phosphorylation in multi-well plates using a phosphospecific antibody, such as with the Envision® (Agilent) system. Further, ELISA-based kits can be developed wherein competition for binding to a phosphospecific antibody is used to assess the state of a particular phosphorylation site, such as with the Alphalisa® format (Perkin Elmer). The relative ease and high throughput of use of these types of assays may be attractive for assays with neuronal cultures because preparation of neuronal cultures can be expensive and time-consuming. Thus, in some embodiments, the level of phospho-S469 CNAα antibody retained by a sample may be measured and visualized using one or more of these detection systems.

A variety of different samples could be used for different antibody-detection platforms. For example, antibody staining can be performed using ex vivo slices from animals or with cultured neurons grown in multi-well plates (such as 96-well or 384-well formats). AlphaLISA® samples can be run with cultured neurons in multi-well plates, such as 32, 48, or 96 well plates.

### Antibody Staining Assays with Brain Slices

Levels of phospho-S469 CNAα can be measured in slices of brain tissue from animals exposed to different treatments or conditions using the phospho-S469 CNAα antibody to measure changes in neuronal activity. For example, the levels of phospho-S469 CNAα could be measured in slices from animals treated and not treated with a specific treatment or condition to determine the change in neuronal activity induced by the treatment or condition. Slices could also be prepared from post-mortem samples from patients with and without neurologic diseases.

The levels of phospho-S469 CNAα can be measured by incubating fixed and blocked slices with the phospho-S469 CNAα antibody and washing off excess antibody and then staining with a labeled secondary antibody that is then washed off. Changes in levels of the secondary antibody could be assessed by immunofluorescence to determine differences in phosphorylation of S469 CNAα at S469 as a measure of neuronal activation.

Alternatively, a wide variety of means could be used to develop directly-labeled antibodies that do not require secondary antibody staining. For example, primary immunofluorescence uses a single, primary antibody that is chemically linked (conjugated) to a fluorophore. Exchange-PAINT is a recent technology can be used to create DNA-barcoded labeling probes that allow super-resolution imaging in situ in fixed cells (see Agasti S S et al., Chemical Science 8:3080-3091 (2017)).

In staining assays, total levels of CNAα and/or a general cellular or neuron marker could be used to standardize measurement of phospho-S469 CNAα over different conditions. In assays to assess levels of phospho-S469 CNAα and one or more other protein, multiple primary antibodies could be used. Primary antibodies from different species (such as rabbit, mouse, etc.) that bind different target proteins can be used with appropriate species-specific secondary antibodies. In this way, levels of phospho-S469 CNAα staining and one or more other signals can be measured in the same sample. Phospho-S469 CNAα staining could also be compared with non-antibody fluorescent measures, such as 4′,6-diamidino-2-phenylindole (DAPI) staining of DNA.

### Antibody Staining Assays with Cultured Neurons

Similar to brain slices, neurons in culture on multi-well plates can be assessed using immunofluorescence after staining for phospho-S469 CNAα and a labeled secondary antibody or staining with a phospho-S469 CNAα antibody conjugated to a detection agent. This can be done with standard microscopes in a qualitative manner and the level of phospho-S469 CNAα staining could be compared to the levels of staining for total CNAα or a marker of neurons, such as MAP2 to mark dendrites.

In addition, fluorescence intensity can also be directly measured using higher-throughput quantitative measures such as Envision multilabel plate readers. In some formats, enzyme-conjugated polymer backbones comprising secondary antibody molecules may be used. The amount of phospho-S469 CNAα staining in cultured neurons on multi-well plates can be assessed directly using a phosphospecific S469 CNAα antibody. For example, changes in the amount of fluorescence produced by phospho-S469 CNAα staining can be compared under different conditions and may be measured in relation to levels of total CNAα staining or a housekeeping gene for neurons.

### Western Blot

Tissue from animal studies or patient samples could also be evaluated by western blot analysis. In this assay, proteins in homogenized tissue from treated animals and human patients could be separated using SDS-PAGE gels. Following transfer to membranes, the phospho-S469 CNAα antibody can be used to measure levels of neuronal activity following similar steps of blocking and primary and secondary antibodies as noted above. The levels of total CNAα and/or a housekeeping gene may be used to normalize values.

### AlphaLISA® Assays

The AlphaLISA® is a bead-based proximity assay that can be run with similar protocols to an ELISA assay (see Eglen R M, et al., Current Chemical Genomics 1:2-10 (2008)). An AlphaLISA® assay can be used with high-throughput fluid dispensing and detection systems and can provide quantitative data using no-wash microtiter plate-based assays.

Bead-based proximity assays, such as AlphaLISA®, are based on high energy irradiation of Donor beads to generate singlet oxygen molecules that can travel over a constrained distance to Acceptor beads. In this format, Donor may comprise a photosensitizing agent (such as phthalocyanine) that when irradiated can excite ambient oxygen to a singlet state. The singlet oxygen can produce measurable chemiluminescence based on excitation of Acceptor beads through a set of chemical reactions. The Acceptor beads may comprise a chemical dye (such as Europium) and activation of these dyes leads to light emission at specific wavelengths (for example 615 nm) that can be easily measured. As singlet oxygen can only travel approximately 200 nm, chemiluminescence following excitation of the reaction mixture is a measure of binding between the agents coupled to the Acceptor and to the Donor beads. However, excitation of a single Donor bead can generate thousands of oxygen singlets per second, leading to a high amplification of the response when this large number of singlets interact with an Acceptor beads in close proximity. A variety of plate readers, such as the Envision® platform, may be used for standard and high-throughput AlphaLISA® formats using laser excitation.

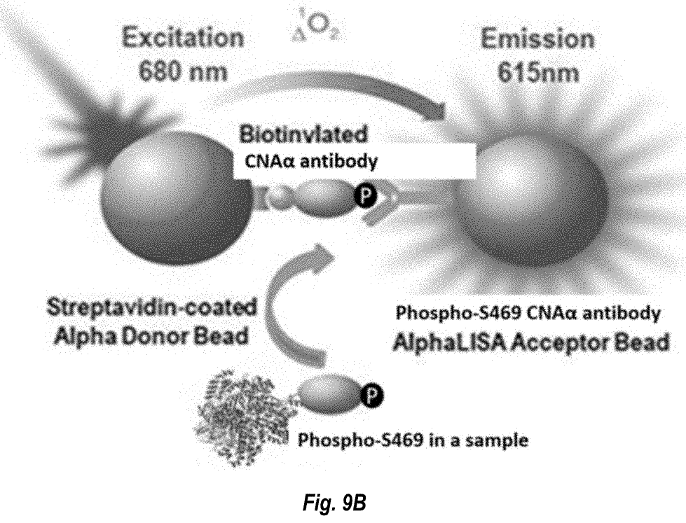

In an example AlphaLISA® assay format, the S469E autoinhibitory domain (AID) peptide may be coupled to Donor beads, while phospho-S469 CNAα antibody is coupled to Acceptor beads, as shown in FIG. 9B. Amino acid substitution of E for S has been well-characterized to mimetic phosphorylation and functionally replace phosphoserine (see Maciejewski P M et al., Journal of Biological Chemistry 270(46):27661-27665 (1995)). Thus, S469E can be considered a phosphomimetic peptide that models phosphorylation of S469. When the Donor beads coupled to S469E AID peptide and Acceptor beads coupled to the phospho-S469 CNAα antibody are added to a sample of tissue containing phospho-S469, this phospho-S469 in the sample can compete the S469E peptide coupled to the Donor beads away from the Acceptor beads coupled to the phospho-S469 CNAα antibody. In other words, the phospho-S469 CNAα present in the sample can reduce the association of the S469E AID peptide and the phospho-S469 CNAα antibody and reduce the level of Donor and Acceptor beads that are bound together and thus reduce chemiluminescence upon activation. Therefore, the amount of phospho-S469 CNAα in a sample could be measured via a competition AlphaLISA® assay, and changes in levels of phospho-S469 CNAα could be measured under a variety of experimental conditions and protocols.

### Fluorescence Resonance Energy Transfer (FRET)

Like AlphaLISA® assays, FRET-based assays can also be used measure changes in phosphorylation at S469 of CNAα. FRET assays are based on the transfer of energy between two fluorophores, a Donor and an Acceptor, which can occur when the donor and acceptor are in close proximity. In this case, a Donor or Acceptor could be grafted covalently to the phospho-S469 CNAα antibody, while the complementary Acceptor or Donor is grafted covalently to the S469E AID peptide. Like the methods of AlphaLISA, the presence of phospho-S469 CNAα in a sample could cause a reduction in FRET signal as the Acceptor and Donor are no longer in close proximity as the S469E peptide is competed away from the phospho-S469 CNAα antibody.

Time-resolved FRET (TR-FRET) methodologies may also be used to decrease background in a sample that is produced non-specifically by buffers, non-selective interaction, etc. Homogenous time resolved fluorescence (HTRF) is an example of an TR-FRET format that is widely used for high-throughput screening.

### Luminex®

Luminex® assays (R&D Systems) can be used for bead-based multianalyte profiling assays. Luminex® assays are run with color-coded beads that are pre-coated with analyte-specific capture antibodies (for example, a phospho-S469 CNAα antibody). Data on samples are generated via biotinylated detection antibodies that are bound to phycoerythrin followed by dual-laser flow-based detection. In addition, Luminex® assays can be used with magnetic beads and read using a MAGPIX® analyzer that captures images of individual well of a multi-well plate.

### High-Content Screening

High-content screening (HCS) can be used to detect changes in cell phenotype based on various treatments. For example, neurons in culture can be incubated with various substances, and changes in the cell phenotype are measured using automated image analysis. For example, fluorescent-labeled phospho-S469 CNAα antibody could be used in HCS platforms to measure changes in neuronal activity based on various treatments. Measurement of phospho-S469 CNAα could be coupled to measurements of changes in cell morphology as well using an HCS platform.

### In-Cell Western

The in-cell western™ (ICW, LICOR) assay is a quantitative immunofluorescence assay that can be performed in microplates, allowing detection of cellular proteins in situ. ICW assays may also be referred to as cytoblots, cell-based ELISA, In-Cell ELISA, and Fast Activated Cell-based ELISA. For example, the ICW format would allow relatively rapid measurement of changes in neuronal activity by various treatments in cultured neurons by quantifying the signal generated with a phospho-S469 CNAα antibody.

### Kits Comprising Phospho-S469 CNAα Antibodies

Also provided herein are kits for measuring the level of phospho-S469 CNAα in a biological sample, wherein the kit comprises phospho-S469 CNAα antibodies and one or more reagents for detecting the level of phospho-S469 CNAα antibodies in the biological sample. In some embodiments, the kits further comprise at least one chemical compound that may alter neuronal activity in a biological sample comprising neurons as a positive or negative control, whereby exposing the phospho-S469 CNAα antibody and the at least one chemical compound to a biological sample comprising neurons to allows for determination of the effect of the at least one chemical compound on activity of the neurons in the sample. Such chemical compounds may include, for example, compounds known to impact neuronal activity, for instance: isoproterenol, brain-derived neurotrophic factor (BDNF), dihydroxypheylglycine (DHPG), N-methyl-D-asparatate (NMDA), bicuculline, strychnine, and tetrodotoxin (TTX), for example. Other potential chemical compounds that may impact activity of neurons include neurotransmitters and modulators listed, for example, in Alexander, SPH & Peters, J A Receptor and Ion Channel Nomenclature Supplement, Trends in Pharmacological Sci. (2000). In some kits herein, the phospho-S469 CNAα antibodies are conjugated to beads. In some kits herein, the reagents for detecting the level of phospho-S469 CNAα antibodies in the biological sample comprise fluorescent or chemiluminescent labels. In some kits herein, the reagents for detecting the level of phospho-S469 CNAα antibodies in the biological sample comprise secondary antibodies and optionally tertiary antibodies. In some embodiments, the kits further comprise instructions for use. In some embodiments, the kits further comprise a substrate for holding a biological sample, such as a multi-well plate.

### EXAMPLES

The following examples are provided to illustrate certain disclosed embodiments and are not to be construed as limiting the scope of this disclosure in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1—Phosphoproteome analysis of chemical LTP (cLTP) Treated Neuronal Cultures

To determine the phosphoproteome of cLTP treated neuronal cultures, a cLTP protocol was developed that optimized treatment of mouse cortical cultures with a no Mg⁺⁺, high glycine buffer stimulation that results in the activation of synaptic NMDA receptors, as previously described in Lu et al., Neuron 9:243-254 (2001).

For mouse cortical cultures, embryos were collected from embryonic day (E)16-17 timed pregnant C57B1/6 mice (Charles River Laboratories) following euthanasia via CO₂. Cerebral cortices were dissected from embryos in dissection medium, comprising Hibernate E (Thermo Fisher Scientific), 2% B27 supplement (Thermo Fisher Scientific), and 2.5% penicillin-streptomycin (Thermo Fisher Scientific). Tissue was washed twice in Hibernate E, prewarmed to 37° C., and digested at 37° C. for seven to eight minutes with papain (Worthington) in Hibernate E, supplemented with 0.01% DNAse (Sigma-Aldrich). Tissue was rinsed three times in prewarmed Hibernate E plus trypsin inhibitor (Worthington) and bovine serum albumin (Sigma-Aldrich) and then triturated with a P1000 pipette in plating medium, consisting of Neurobasal (Thermo Fisher Scientific), 5% B27, 100 μM glutamax, and 10% fetal bovine serum (Life Technologies), plus 0.01% DNAse. Cells were centrifuged for five minutes at 1,000 RPM, resuspended in plating medium, and plated in poly-d-lysine coated 10 cm plates at 6 million cells per plate (VWR). Cultures were grown in a 5% CO₂ incubator. Twenty-four hours following plating, plating medium was replaced with NbActiv1 medium (BrainBits), and 50% of medium was replaced every 3-4 days.

The chemical long-term potentiation (cLTP) protocol was modified as described in Lu et al. 2001. Mouse cortical cultures at 15 days in vitro (15DIV) in 10 cm plates were pretreated for 25 min with artificial cerebral spinal fluid (aCSF) supplemented with bicuculline (20 μM), strychnine (3 μM), and tetrodotoxin (0.5 μM) (equilibration buffer) and then washed once in aCSF plus bicuculline (20 μM) and strychnine (3 μM). Control cultures were then incubated for 10 min in equilibration buffer and cLTP-treated cultures in aCSF plus bicuculline (20 μM), strychnine (3 μM), and glycine (200 μM). In all cases, buffer was then replaced with equilibration buffer, and cells were incubated for 5, 20, 40, and 60 min.

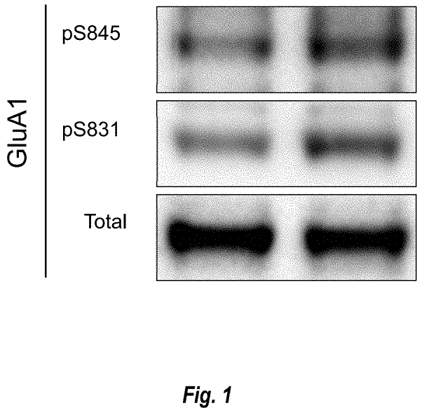

This stimulation resulted in the phosphorylation of the GluA1 AMPA receptor subunit on S831 and S845 as previously described (FIG. 1). (Diering G H et al., Neuron 84:790-805 (2014); Lee HK et al., Nature 405: 955-959.]

Next, mouse cortical neurons were treated at 15DIV grown in 10 cm plates with this buffer or a control buffer for 10 minutes, restored to the control buffer, and allowed to recover for different periods of time, namely 5, 20, 40, and 60 min. Neurons were harvested and mass spectrometry was performed using 10-plex tandem mass tag (TMT) methodology for total and phosphoproteome changes following these treatments, using two independent sets of cortical cultures.

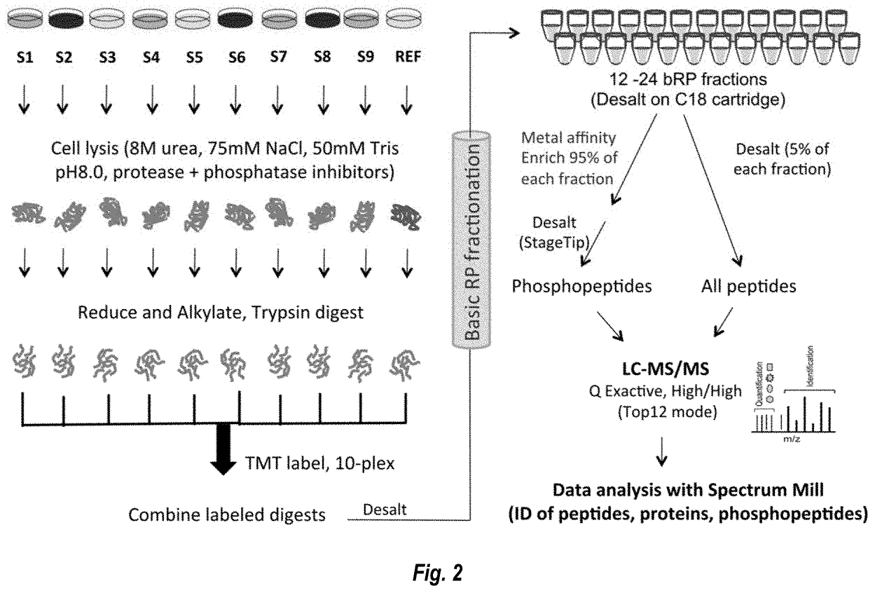

FIG. 2 outlines the workflow of the phosphoproteome experiment. The pipeline shown is equally applicable to 4-plex iTRAQ, 6-plex TMT or 10-plex chemical labeling for relative quantification; the 10-plex TMT labeling method is illustrated. For mass spectrometry experiments, cells were washed twice in ice-cold PBS, and cells were scraped into Eppendorf tubes and centrifuged at 1800 RPM for 5 min at 4° C. Residual PBS was aspirated, and pellets were frozen at −80° C. until analysis. Proteins were extracted with urea buffer, consisting of 8 M urea (Sigma-Aldrich), 75 mM NaCl (Sigma-Aldrich), 50 mM Tris pH 8.0 (Ambion), 1 mM EDTA, 2 μg/mL Aprotinin (Sigma Aldrich), 10 μg/mL Leupeptin (Roche), 1% phosphatase inhibitor cocktail 2 & 3 (Sigma-Aldrich), 10 mM NaF (Sigma-Aldrich), and 1 mM PMSF (Sigma Aldrich). Proteins were digested with LysC/trypsin, and peptides from each sample are labeled with 10-plex TMT chemical mass tag reagents (Thermo Fisher Scientific) for relative quantification. Following labeling, peptides were combined and fractionated by basic reversed-phase (bRP) chromatography to decrease sample complexity and increase the dynamic range of detection. The global proteome of each plex was measured with 24 bRP fractions using 60 h (24×2.5 h runs) of measurement time on a Q Exactive Plus instrument (Thermo-Fisher). For phosphoproteome analysis, phosphorylated peptides were enriched with immobilized metal affinity chromatography (IMAC) and injected as 12 LC-MS/MS runs requiring 30 h measurement time per plex.

All mass spectrometry data were analyzed using Spectrum Mill (SM). In SM, false discovery rates (FDRs) are calculated at 3 different levels: spectrum, distinct peptide, and distinct protein. Peptide FDRs were calculated in SM using essentially the same pseudo-reversal strategy evaluated by Elias and Gygi Methods Mol Bio 604:55-71 (2010). A false distinct protein identification (ID) occurred when all the distinct peptides that group together to constitute a distinct protein have a deltaForwardReverseScore≤0. Settings were adjusted to provide peptide FDR of 1-2% and protein FDR of 0-1%. Only proteins with >2 peptides and at least 2 TMT ratios in each replicate were counted as being identified and quantified.

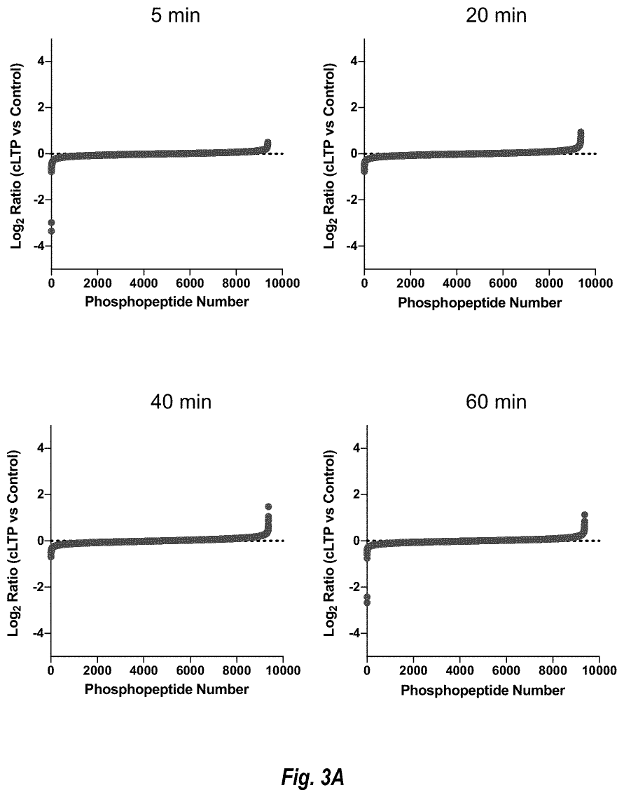

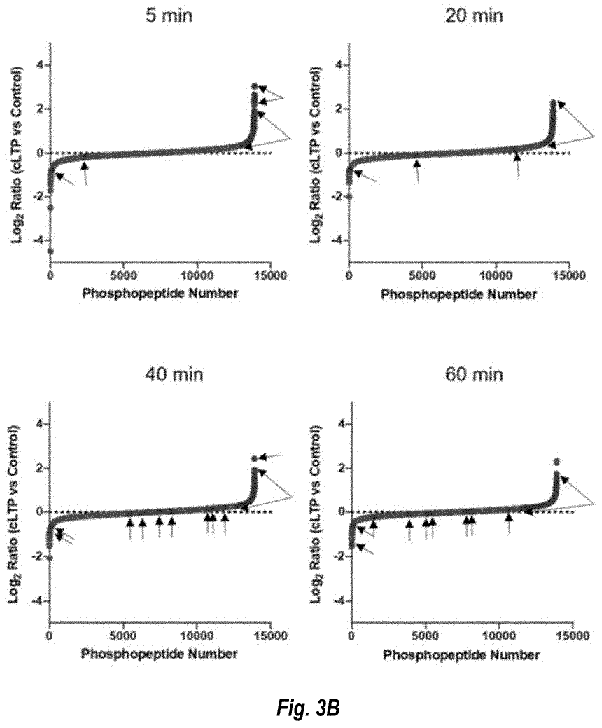

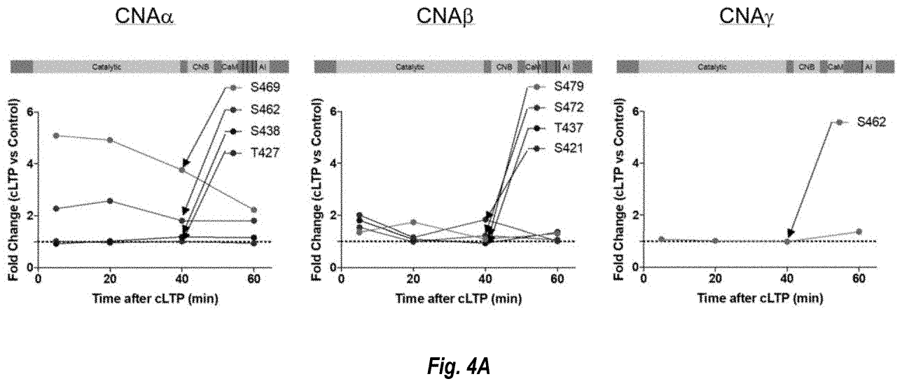

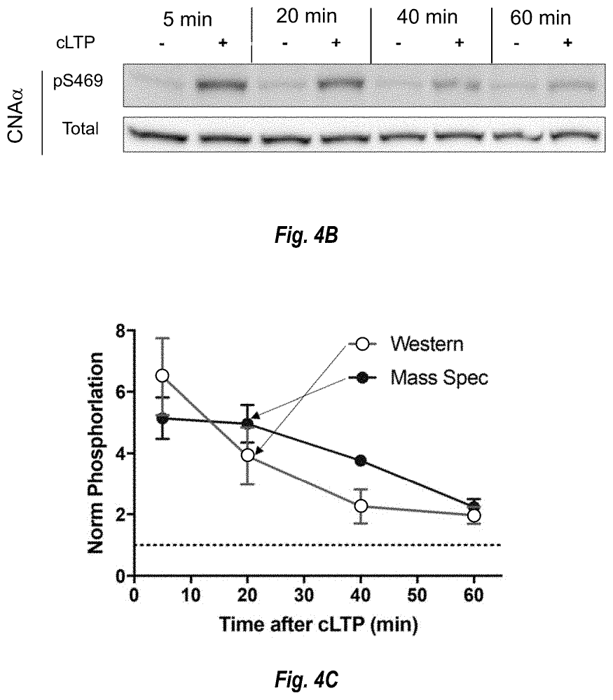

For the total proteome, 9371 individual proteins were identified in all four conditions and both experimental replicates. As seen in FIG. 3A, there were no significant changes in the level of any individual protein following these treatments. When the phosphoproteome was analyzed, there were 13,904 phosphopeptides identified in each condition and in both experimental replicates. Of these, 60 were significantly regulated following cLTP treatment, as shown by the arrows in FIG. 3B. The most significantly regulated phosphopeptide was S469 from the protein encoded by Ppp3ca gene, the alpha paralog of the catalytic subunit of calcineurin (CNAα), a residue at the N-terminal end of the calcineurin autoinhibitory domain (AID). The S469 phosphorylation site showed fold-changes of +5.10, 4.92, 3.76, and 2.22 relative to controls at 5, 20, 40, and 60 min following cLTP treatment, respectively (FIG. 4A). While S462 from CNAα showed a trend towards an increase in phosphorylation at all time points following cLTP, no other phosphorylation site was altered on CNAα or its β or γ paralogs, including the equivalent sites as S469, which are S479 from CNAβ and S462 from CNAγ, respectively.

A custom rabbit polyclonal phosphospecific antibody was developed against CNAα. For the phosphospecific S469 CNAα antibody, rabbit polyclonal antiserum was developed against the peptide PQHKIT[pS]FEEA (SEQ ID No: 1), and antiserum was affinity purified using phosphorylated peptide and negatively selected against the non-phosphorylated version of the peptide (21^{ST} Century Biochemicals). For the total CNAα antibody, rabbit polyclonal antiserum was developed against the following peptide RRDAMPSDANLNSINK (SEQ ID No: 2). Antiserum was affinity purified using the immune peptide (Open Biosystems).

For western blotting, primary mouse cortical neurons in 10 cm plates were treated with the cLTP protocol, rinsed once with ice cold PBS, and then lysed with 500 μl of urea buffer as described above, supplemented with protease inhibitors (Roche), phosphatase inhibitors (Sigma-Aldrich), NaF (Sigma-Aldrich), okadaic acid (EMD Millipore), and PMSF (Sigma-Aldrich). Cells were scraped into an Eppendorf tube and centrifuged at 14,000 RPM for 15 min at 4° C., and supernatant transferred to a new Eppendorf tube. Protein concentrations were quantified with a BCA kit (Pierce). Proteins were heated in NuPAGE loading buffer (Invitrogen) plus NuPAGE sample reducing agent (Invitrogen) at 95° C. for 10 min. For each lane, 10 μg protein was loaded. Protein was run on 4-12% Bis-tris Bolt gels (Invitrogen) and transferred to nitrocellulose filters using a iBlot2 transfer system (Thermo Fisher Scientific). Membranes were blocked in TBS-T (Thermo Fisher Scientific) plus 5% BSA (Sigma-Aldrich) for 1 hour at room temperature and then primary antibodies overnight in blocking buffer at 4° C. Membranes were washed in TBS-T, incubated in secondary antibodies for 1 hour at room temperature, washed in TBS-T, and then imaged with ECL (Thermo Fisher Scientific) on a ChemiDoc MP imaging station (Biorad). For purified enzymes, westerns were performed as above, using 0.1 μg of purified protein. Rabbit anti-GAPDH (Cell Signal Technology, 1:1000) was a commercially-available primary antibody. Donkey-anti-Rabbit-HRP (GE Healthcare) was the secondary antibody.

When lysates from primary mouse cortical cultures treated with the cLTP protocol were probed with this phosphospecific antibody, an increase in S469 CNAα phosphorylation at 5 min post-cLTP was seen that reduced over time towards baseline after 60 min (FIG. 4B). This pattern is consistent with the quantification observed with the mass spectrometry data (FIG. 4C). Normalized phosphorylation refers to the change relative to neurons treated with a control buffer. These data confirm the mass spectrometry results showing cLTP-dependent induction of S469 CNAα phosphorylation and demonstrate that this custom phosphospecific antibody can be used as a tool for measuring cLTP-related phosphorylation of calcineurin.

### Example 2—Characterization of Effects of Phosphorylation of CNAα S469 on Enzyme Function

The calcineurin holoenzyme is comprised of a ˜56 kD catalytic subunit (CNA) and a ˜25 kD regulatory subunit (CNB). There are three paralogs of the catalytic subunit, PPP3CA, PPP3CB, and PPP3CC, that encode the α, β, and γ isoforms, respectively. The catalytic subunit is comprised of four domains, a catalytic domain, a regulatory subunit binding domain, a calmodulin (CaM) binding domain, and the autoinhibitory domain (AID). As seen in FIG. 5A, the AID wraps to sterically block the catalytic site of the enzyme, and the binding of active CaM reduces that autoinhibition, substantially enhancing phosphatase activity. Since S469 of CNAα resides at the N-terminal end of the AID, its phosphorylation might be predicted to alter enzyme activity by modulating the binding of the AID to the catalytic site.

Analysis of published reports of the calcineurin crystal structure indicates that S469 sits opposed to D285 within the catalytic site, as shown in FIG. 5A. See Kissinger C R et al., Nature 378: 641-644 (1995). Therefore, phosphorylation of S469 might be predicted to result in a steric hindrance between the phosphorylated serine and D285, thus decreasing the ability of the AID to block the enzyme and increasing calcineurin function, as shown in FIG. 5B.

To investigate whether phosphorylation of CNAα Ser469 modifies calcineurin phosphatase activity, a phosphomimetic mutant was generated by replacing S469 with E469 in purified, recombinant human calcineurin. The S469E mutant or wild type of CNAα was co-expressed with CNB1 subunit in E. coli and co-purified. Both mutant and wild type show similar expression level and purity. Their concentrations were quantified by OD280 and CaM titration.

Enzyme activity was then assessed. CN enzymatic parameters for wild-type and S469E mutant proteins were measured using phosphorylated RII (pRII) peptide in a buffer containing 50 mM HEPES (pH=7.0), 1 mM CaCl₂, 1 mM MnCl₂, 0.5 mM dithiothreitol (DTT), and 0.1 mg/ml bovine serum albumin (BSA). Phosphate product release was monitored at 620 nm on SpectraMax_i3x spectrophotometer (Molecular Devices) with a 96-well plate-based calcineurin phosphatase assay kit (Abcam). Briefly, CN (40 nM) and CM (200 nM) were pre-mixed in 2× assay buffer. The reaction was initiated by addition of 25 μl pRII substrate (in H₂O) to equal volume of CN/CaM mixture. The final concentration of pRII used ranged from 12 μM to 750 μM. Reaction was stopped by green quench reagent after 20 minutes. Kₘ was determined using nonlinear regression of the Michaelis-Menten equation.

For the determination of IC50 values for phospho-AID peptides, to a 25 μl solution mixture containing recombinant CN (40 nM) and CaM (200 nM), 10μl AID or phospho-AID peptide was added to final concentrations ranging from 1.5 μM to 100 μM. After incubation at room temperature for 10 min, 15 μl pRII substrate (500 μM) was added to initiate the reaction. Final product was monitored at OD_{620 nm} on SpectarMax_i3x and IC50 values were derived from non-linear fitting of Logistic equation using Origin8.

The specific activity for the phosphomimetic S469E in the presence of CaM is higher due to a lower Km (WT: 610 μM; S469E: 256 μM, FIG. 5C). These experiments suggest that S469 is important for interaction of the AID with calcineurin catalytic domain and that phosphorylation at S469 enhances calcineurin phosphatase activity.

### Example 3—Characterization of Neuronal Activity on Phosphorylation of CNAα S469

The subcellular localization of phospho-S469 CNAα within neurons was investigated following stimulation.

For rat hippocampal cultures, embryos were collected from E18-19 timed pregnant Sprague-Dawley rats following euthanasia via CO₂. Hippocampi cortices were dissected from embryos, and neurons were cultured as above for mouse cultures in 96-well poly-D-lysine plates (VWR), except that culture media was NbActiv1 (BrainBits) and media was not changed following plating.

Rat hippocampal cultured neurons were grown in 96-well plates, treated with the control or cLTP protocol as described above for mouse cultures, and stained for phospho-S469 CNAα and for MAP2 to mark dendrites.

For immunostaining, neurons grown in 96-well plates were fixed in 4% paraformaldehyde/4% sucrose for 15 minutes at room temperature, washed three times in PBS, permeabilized in 0.25% Triton X-100 for 10 min at room temperature, and then blocked with 5% BSA for 1 hour at room temperature. They were incubated in primary antibodies diluted in 1% BSA at 4° C. overnight, rinsed three times in PBS, and incubated in secondary antibodies diluted in 1% BSA for 1 hour at room temperature. When applicable, Hoechst33342 (10 μg/mL; Thermo Fisher Scientific) was added for 3 min at room temperature. Cultures were then rinsed three times in PBS and stored at 4° C. prior to imaging. Additional primary antibodies were as follows: mouse anti-synapsin1 (Synaptic Systems, 5 μg/mL) and chicken anti-MAP2 (Abcam, 1:2000). Secondary antibodies were as follows: goat anti-rabbit Alexa 488 (Thermo Fisher Scientific, 4 μg/mL); goat anti-mouse Alexa 568 (Thermo Fisher Scientific, 4 μg/mL); and goat anti-chicken Alexa 647 (Thermo Fisher Scientific, 4 μg/mL).

Images were acquired on Opera Phenix® high-content confocal imaging system (Perkin Elmer) equipped with a 63×b, water objective, sCMOS cameras and the Harmony® high-content imaging and analysis software. All the image analysis was done by using Columbus® image data analysis system (Perkin Elmer).

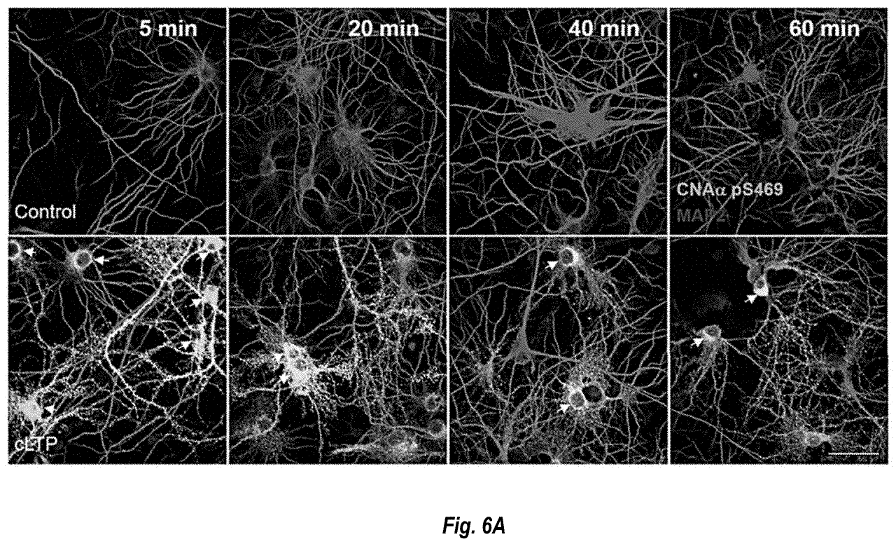

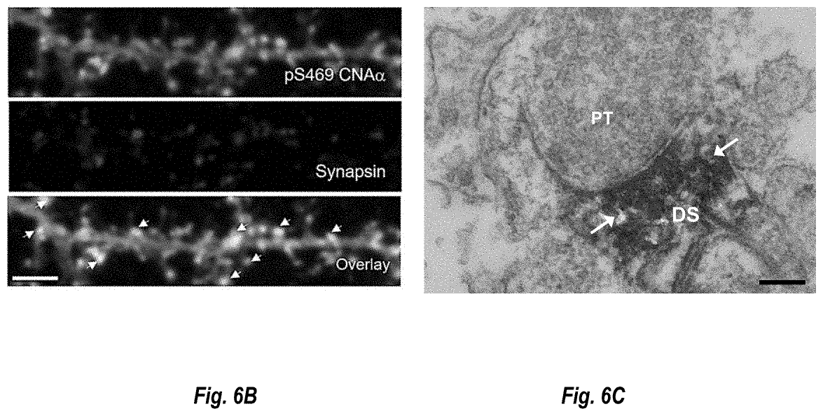

Confocal imaging using a Phenix® high content plate reader was performed and showed a dramatic increase in phospho-S469 CNAα signal within neurons at 5 min following cLTP (as shown by arrows indicating neurons positive for both MAP2 and phospho-S469 CNAα) that returns towards baseline by 60 min (FIG. 6A).

Higher magnification images of cLTP treated rat hippocampal neurons stained for phospho-S469 CNAα and synapsin shows an enrichment of phospho-S469 CNAα at apparent synaptic contacts (with arrows in overlay panel indicating regions of colocalization of phospho-S469 CNAα and synapsin in FIG. 6B).

Immuno-electron microscopy was also performed. Cells were fixed in 2.5% glutaraldehyde, 3% paraformaldehyde with 5% sucrose in 0.1M sodium cacodylate buffer (pH 7.4), scraped and pelleted in an Eppendorf tube, washed with 0.1M sodium cacodylate buffer 3×, and post-fixed in 1% OsO4 in veronal-acetate buffer 1 hour. The pellet was stained in block overnight with 0.5% uranyl acetate in veronal-acetate buffer, and then dehydrated and embedded in Embed 812 resin. Sections were cut on a Leica Ultracut UCT microtome with a Diatome diamond knife at a thickness setting of 50 nm, stained with uranyl acetate, and lead citrate. The sections were examined using a FEI Tecnai spirit at 80 KV.

Analysis using immuno-electron microscopy of synapses from rat hippocampal cultures shows that phospho-S469 CNAα is predominantly localized to dendritic spines (FIG. 6C).

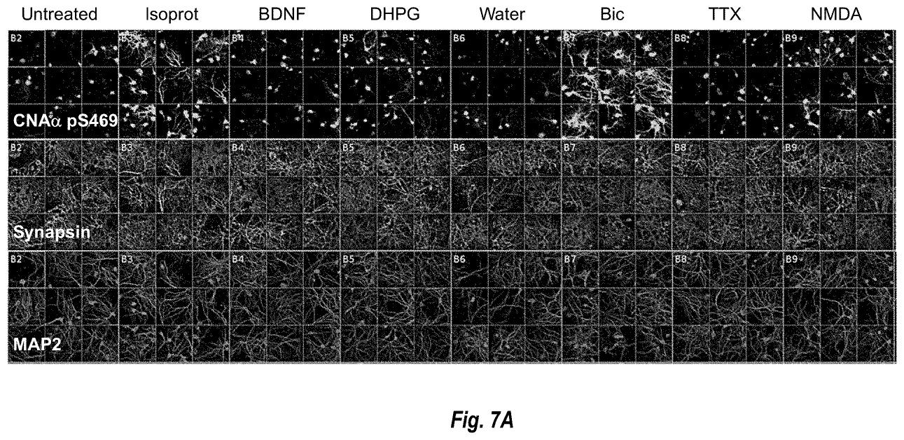

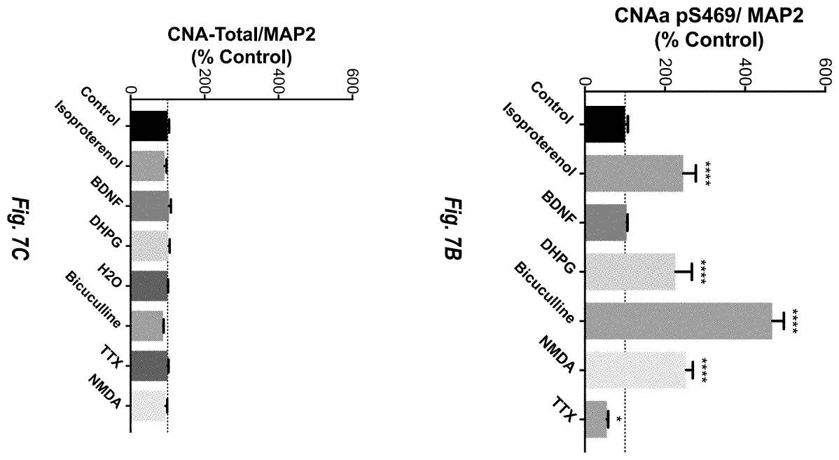

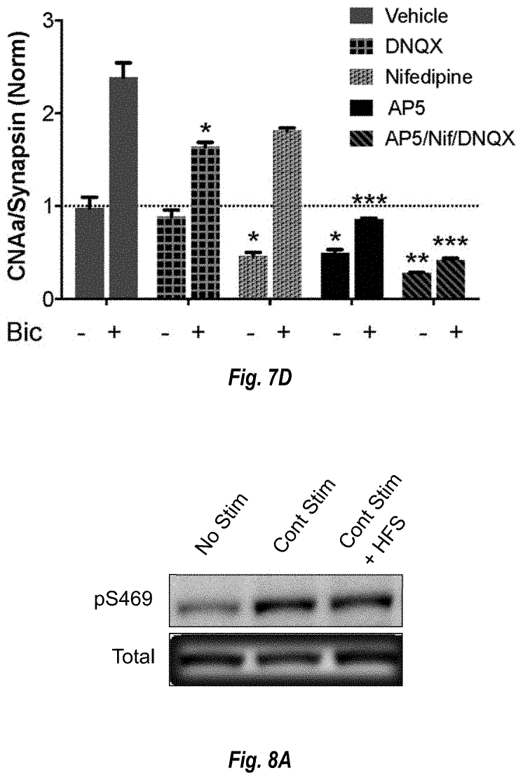

The 96-well assay format was also used to further define the stimuli that induce CNAα phosphorylation on S469. Neurons grown in 96-well plates were treated with various stimuli known to activate or inhibit neuronal activity and forms of plasticity, including 2 μM isoproterenol, 50 μg/mL brain-derived neurotrophic factor (BDNF), 100 μM dihydroxypheylglycine (DHPG), 20 μM N-methyl-D-asparatate (NMDA), 20 μM bicuculline, and 0.504 tetrodotoxin (TTX), and stained for phospho-S469 CNAα and MAP2 (FIG. 7A, with quantification in FIG. 7B). Agents known to increase neuronal activity, i.e., isoproterenol, DHPG, NMDA, and bicuculline, all significantly and robustly increased the levels of phospho-S469 CNAα within dendrites, while TTX, which blocks neuronal activity, caused a decrease in phospho-S469 CNAα. In contrast, BDNF, a growth factor stimulus, showed no change in phospho-S469 CNAα levels. In all cases, there were no changes in total CNAα levels or in dendrites (FIG. 7C).

Next, the dependency of S469 CNAα phosphorylation was assessed when neurons were treated by blockade of glutamate receptors and L-type Ca channels using pharmacological blockers. DNQX is an AMPA and kainate glutamate receptor antagonist, AP5 is an NMDA glutamate receptor antagonist, and nifedipine is an L-type calcium channel blocker.

Inhibition of AMPA and NMDA receptors and L-type Ca2+ channels caused a reduction in S469 CNAα phosphorylation, with NMDA receptor inhibition (by AP5) most strongly reducing baseline and bicuculine-induced S469 CNAα phosphorylation (FIG. 7D). In total, these data show that the levels of phospho-S469 CNAα within neurons were highly sensitive to neuronal activity levels and that the phospho-S469 CNAα antibody can be used as a readout of neuronal activity levels of neurons. In combination with the enzyme data, these results indicate that a recent history of activity increases S469 CNAα phosphorylation within neurons.

Next, potential induction of S469 CNAα phosphorylation by stimulation in mouse brain tissue was determined slice cultures. For slice cultures, 6-week-old in-house-bred C57B1/6 male mice were deeply anesthetized with isoflurane and sacrificed by decapitation. The brain was then rapidly removed and placed into ice-cold and oxygenated (95% O₂/5% CO₂) cutting solution (in mM: 194 Sucrose, 30 NaCl, 4.5 KCl, 1.2 NaH₂PO₄, 26 NaHCO₃, 10 Glucose, 2 MgCl₂, 0.2 CaCl₂). A vibratome (Leica VT1200S) was used to cut 400 μm-thick coronal sections. Dorsal and intermediate portions of the hippocampi of both hemispheres were then isolated from the rest of the brain. Finally, the dentate gyrus, CA3 region were removed from each slice by manual dissection to obtain “mini-slices” containing the CA1 region. A small portion of the molecular layer of the dentate gyrus near the hippocampal fissure was left attached to avoid cutting the apical dendrites of the CA1 pyramidal cells.

Mini-slices were then kept in a chamber with artificial cerebrospinal fluid or aCSF (in mM: 119 NaCl, 2.3 KCl, 1 NaH₂PO₄, 26 NaHCO₃, 11 Glucose, 1.3 MgCl₂, 2.5 CaCl₂) to recover for a minimum 2 hours at 32° C. After recovery, they were transferred to a submersion chamber containing heated aCSF (˜28-30° C.) and a bipolar stimulation electrode was placed on the schaffer collaterals. A subset of slices were not stimulated and served as the control condition (unstimulated). Another subset only received baseline stimulations at half maximal intensity (ranging from 200 to 400 μA) at 0.05 Hz for approximately 4 min. Finally, a subset of mini slices were stimulated with high frequency stimulations (HFS, 4 trains of 1 s long 100 Hz stimulations) following baseline stimulation. Potentiation of field responses from the apical dendrites of the CA1 region were recorded post-HFS using a glass pipette (2-3 mOhm). Mini-slices were collected 5 min after control stimulation, HFS, or no stimulation, and snap-frozen by transferring them into 1.5 mL microcentrifuge. Mini-slices that failed to show at least 150% increase in their responses following HFS were discarded immediately. In some cases, the mini-slices were prepared after stimulations. In total, 17 slices from 4 animals were collected and 5-6 slices were used for each condition. Mini-CA1 hippocampus slices were then homogenated in lysis buffer (8M urea buffer supplemented with protease and phosphoprotease inhibitors on ice). Protein concentration was measured by running BCA assay (Thermo Fisher Scientific) and 10 μg protein was loaded onto 4-12% Bis-tris Bolt gels (Thermo Fisher Scientific) for western blot analysis.

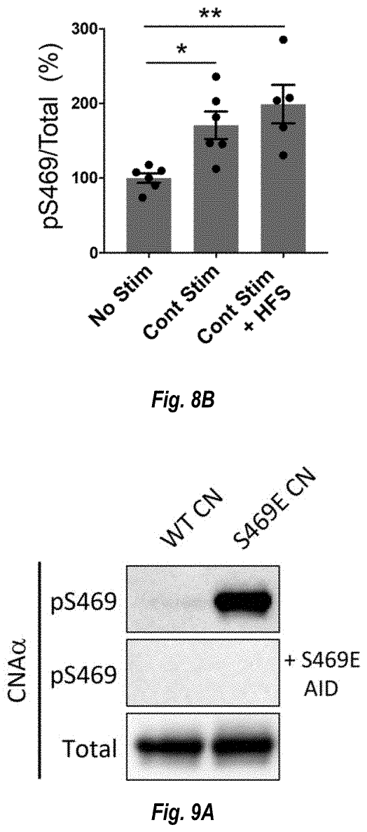

Thus, isolated ventral hippocampal CA1 mini-slices were compared following activation activated with field stimulation, using the following patterns: 1) no stimulation control, 2) 0.05 Hz control baseline stimulation, or 3) 0.05 Hz control baseline stimulation followed by a high frequency stimulation that induces LTP. The control baseline stimulation caused a significant increase in S469 CNAα phosphorylation that was not increased further by the LTP stimulation (FIG. 8A, with quantification of pS469/total CNAα signal in FIG. 8B).

These data indicate that S469 CNAα is phosphorylated by stimulation in hippocampal slices. Furthermore, phosphorylation of S469 CNAα is induced by low frequency stimuli and does not require full LTP stimulation, showing that phosphorylation of S469 CNAα marks stimulated, active neurons. Lastly, the culture and slice data combined demonstrate that the S469 CNAα phosphospecific antibody can be used to mark the activity level of neurons in processed cellular or tissue samples.

### Example 4—Blockade of phospho-S469 CNAα Antibody by a CNAα S469E AID Peptide

It was investigated whether binding of the phospho-S469 CNAα antibody could be blocked by an AID peptide of CNAα containing a S469E mutation.

Recombinant wild-type and S469E CNAα were assessed with a total and phospho-S469 CNAα antibody via western blot. In one condition, the phospho-S469 CNAα antibody was preincubated with the S469E peptide. For “+S469E AID” the CN AID peptide with a S469E mutation was preincubated with the pS469 polyclonal antibody at 1 μM for 30 min. The phospho-S469 CNAα antibody recognized only the S469E CNAα recombinant protein, and the S469E peptide blocked the ability of the phospho-S469 CNAα antibody to do so (FIG. 9A). These data support the potential for the development of ELISA-based and other kits for the measurement of the level of phospho-S469 CNAα in cellular or tissue samples.

FIG. 9B shows a non-limiting example of the design of an ELISA-based kit using the Alphalisa format (Perkin Elmer), wherein the complementary beads of the kits are conjugated to the phospho-S469 CNAα antibody and the S469E AID peptide, respectively. To measure levels of phospho-S469 in a sample (shown at bottom of FIG. 9B), the samples of interest would be added to an assay containing these beads, and an increase in phosphorylation levels would be measured as an increase in the Alphalisa signal. In some embodiments, the complementary beads of the kits are conjugated to an immunogen peptide with a S469E mutation. To measure levels of phosph-S469 in a sample, the samples of interest would be added to an assay containing these beads, and an increase in phosphorylation levels would be measured as a decrease in the Alphalisa signal. Since the phospho-S469 antibody marks the activity level of neurons, such an assay could be used to measure the effect of perturbations, such as compounds or genetic manipulations, on the activity of neurons in a high-throughput format.

### Example 5—Characterization of Phospho-S469 CNAα Levels in Neurons with a Knockdown of Autism-Related Genes

The effect of knockdown of autism-related genes on phosphor-S469 CNAα was also evaluated.

For this study, the autism-related genes Fmr1, Syngap1, Pten, and Akap9 were chosen for evaluation of knockdown of protein levels by small interfering RNA (siRNA). The siRNAs were Accell SmartPools (GE Healthcare), added at 1 μM on 7DIV to rat hippocampal cultures in 96-well plates. siRNAs targeting the autism-related genes, Fmr1, Syngap1, Pten, and Akap9, as well as GSK3a as a negative control, were used.

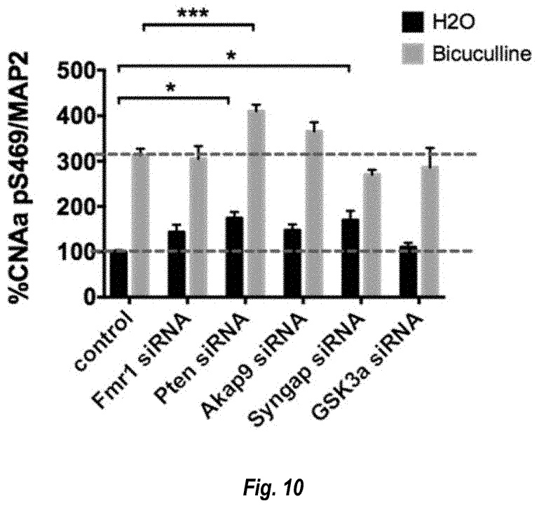

At 21DIV, neurons were stimulated with either bicuculline for 5 min at 20 μM or a vehicle control (water). Neurons were then fixed and stained for MAP2 and phospho-S469 CNAα, with quantification of percent CNAα pS469/MAP2 staining presented in FIG. 10.

There was a significant increase in basal S469 CNAα phosphorylation for Pten and Syngap1 knockdown in water-treated samples and in S469 CNAα phosphorylation in bicuculline-treated samples for Pten knockdown. These data indicate that increases in neural activity levels are measurable by increases in S469 CNAα phosphorylation following reduction of Pten and Syngap1 levels, consistent with reports in tissue from knockout mice (see Clement J P et al, Cell 151(4): 709-723 (2012) and Williams M R et al, J Neurosci. 35(3): 943-959 (2015)).

### Example 6—cLTP Assays with a Monoclonal Mouse Anti-pS469 CNAα Antibody

A monoclonal mouse anti-pS469 CNAα antibody with the heavy and light chain variable region amino acid sequences of SEQ ID Nos: 8 and 10 shown in the Sequence Table below was prepared. This antibody was, accordingly, tested in the cLTP assays and for specificity for phosphorylation at S469.

A cLTP assay was performed using the monoclonal mouse anti-pS469 CNAα antibody, to determine if this antibody can be used to measure cLTP-related phosphorylation of calcineurin. (See, e.g., Example 1 above and FIGS. 4B and 4C.) Western blotting was performed on primary mouse cortical neurons treated with the cLTP protocol as described in Example 1 above. Specifically, Western blot of protein extracts from control and glycine-treated cLTP cultured cortical neurons were probed with the anti-pS469-CNAα antibody and with an anti-CNAα rabbit antibody (to provide total CNAα). Protein lysates were prepared using an 8 M urea solution at 5, 20, 40 and 60 min subsequent to cLTP induction.

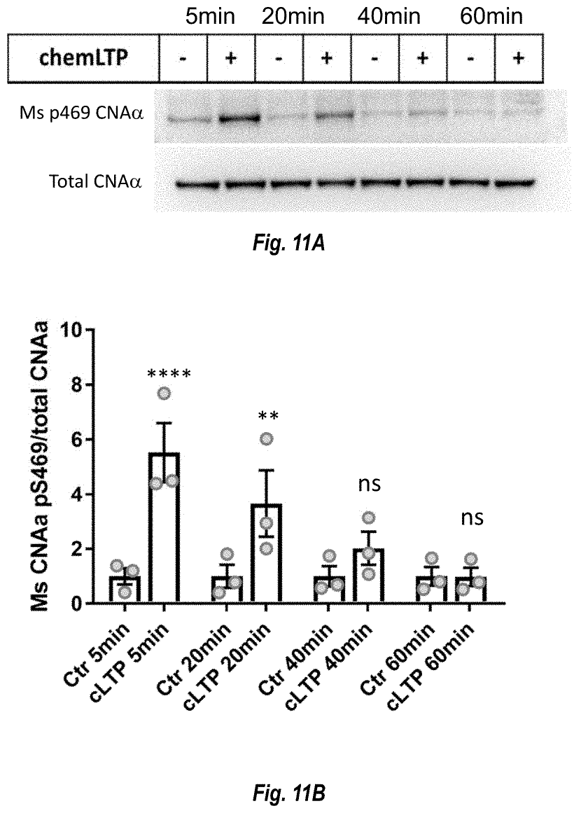

Results are shown in FIG. 11A. Like FIG. 4B, FIG. 11A shows that the antibody is suitable for measuring cLTP-related phosphorylation of calcineurin, and that CNAα phosphorylation increases at 5 minutes post cLTP and then reduces over time towards baseline at 60 minutes post cLTP.

FIG. 11B shows a quantification of the Western blot results by densitometry, comparing the phosphorylated vs. total CNAα signal intensities at 5, 20, 40 and 60 min subsequent to cLTP induction compared to the non-cLTP controls. The data show a 5.52-fold and 3.66-fold increase of phosphorylation at S469 5 and 20 minutes post-cLTP induction, respectively. The densitometric analysis of the immunoblots was performed using the program ImageJ. Data shown are averages of three independent experiments, results of which are shown as open circles on the bar plots. P-values were calculated from a one-way ANOVA with Sikak post hoc test, and ****indicates P<0.0001 while ***indicates P<0.001.

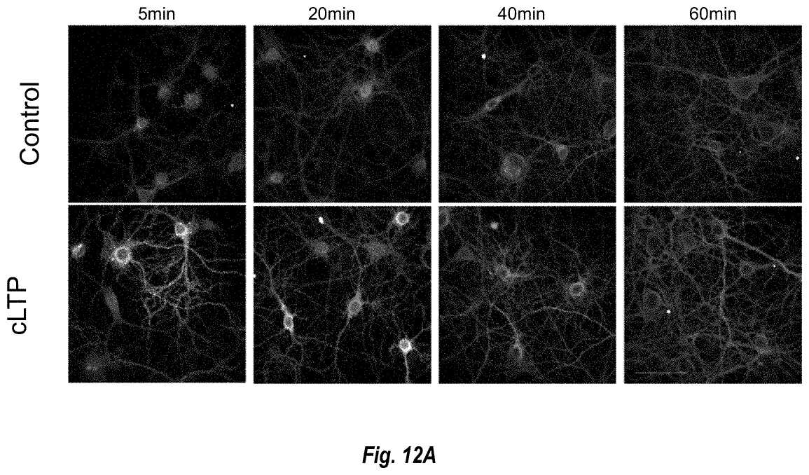

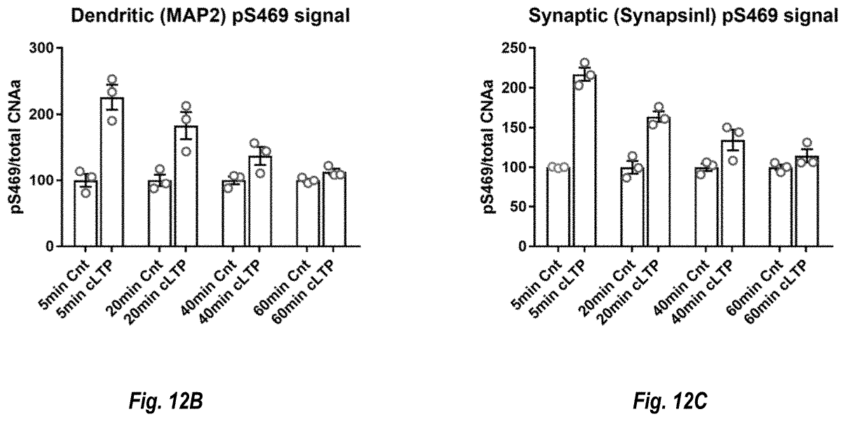

The antibody was further used to visualize cLTP induction in an immunocytochemistry analysis, as shown in FIGS. 12A, B, and C. The protocol was similar to that described in Example 3 above. Immunofluorescence images of hippocampal neurons (21DIV) were taken using an Opera Phenix® high content screening system with Harmony® software and 63× magnification. As shown in FIG. 12A, images were taken of cells 5, 20, 40, and 60 minutes after cLTP induction and of control cells at the same time-points. The anti-pS469 antibody was stained in green. The synaptic marker synapsinI and the dendritic marker MAP2 were also stained (not shown in FIG. 12A).

Quantitation of the staining data is shown in FIGS. 12B and 12C. FIG. 12B quantifies intensity of the antibody co-localized with synapsinI to evaluate changes in S469 phosphorylation on synapses. After 5 and 20 minutes post-cLTP, synaptic-located antibody signal increased 216.8% and 163.8% respectively (expressed as percentage of control values). FIG. 12C quantifies intensity of the antibody co-localized with MAP2. The co-localized staining increased by 225.8% and 183.1% after 5 and 20 minutes post-cLTP, respectively (expressed as a percentage of control values).

Data in FIGS. 12B and C are averages of three separate experiments as shown by the open circles on the bar plots. Harmony® software and maximum projection images were used to perform the analysis. These figures show that the phosphorylation increases within 5 minutes of cLTP induction then falls toward baseline at 60 minutes post induction.

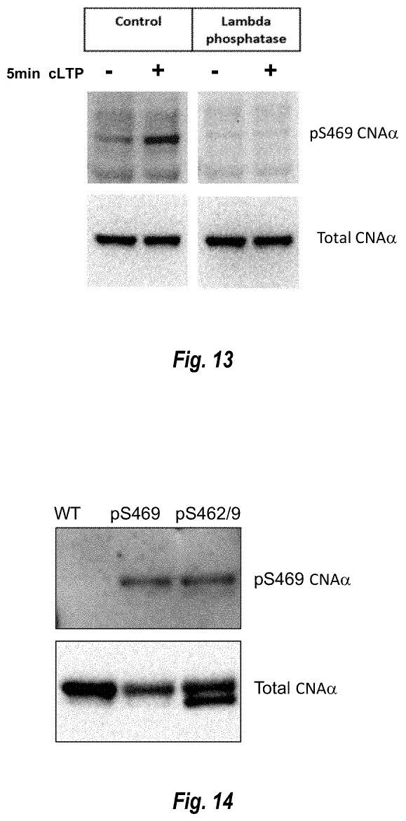

To further test the specificity of the murine monoclonal antibody for phosphorylated CNAα, de-phosphorylation experiments were performed using lambda phosphatase on protein fixed in a membrane. Results are shown in FIG. 13. Specifically, protein lysates from cortical neurons (15DIV), either 5 min post-cLTP induction or no cLTP control, were separated by SDS-PAGE and subsequently transferred to a PVDF membrane. One section of the membrane was incubated overnight at room temperature with lambda phosphatase (FIG. 13, top panel, right blot), while another section was incubated under the same conditions without phosphatase (FIG. 13, top panel, left blot). Following these incubations, the phosphatase-treated and control membrane sections were incubated with the antibody and antibody binding was detected by chemiluminescence. This phosphospecify test shows that the monoclonal antibody is only able to recognize CNAα in absence of lambda phosphatase treatment, i.e. it can only recognize phosphorylated CNAα. As a loading control, the level of total CNAα was also assessed in both membranes sections after the incubations with or without phosphatase (bottom panel of the figure).

The validation of the specificity of the antibody for pS469 was additionally determined by Western blot using CNAα recombinant protein WT (unphosphorylated), the phosphomimic mutant S469E (see examples above) and a double mutant with substitutions of E for S at both positions 462 and 469 (S462E/S469E). Immunoblots (top panel of FIG. 14) show that the antibody recognizes the recombinant protein S469E but not the wild-type unphosphorylated protein. In addition, the recognition of the double mutant S462E/S469E suggests that the antibody is able to recognize S469E even in presence of the S462E mutant. Levels of total CNAα were used as a loading control and were assessed after stripping of the membranes and incubation with anti-CNAα rabbit by chemiluminescence (bottom panel).

The section headings used herein are for organizational purposes only and are not to be construed as limiting the subject matter described. All references cited herein, including patent applications and publications, are incorporated herein by reference in their entireties for any purpose.

### DESCRIPTION OF SEQUENCES

The following table provides a listing of certain sequences referenced herein.

| Description | Sequence | SEQ ID NO |
|---|---|---|
| Amino acid | PQHKIT[pS]FEEA | 1 |
| sequence of a | | |
| phosphospecific | | |
| S469 CNAα antibody | | |
| epitope, wherein | | |
| [pS] refers to | | |
| phosphorylated | | |
| serine | | |
| Amino acid | RRDAMPSDANLNSINK | 2 |
| sequence of a pan | | |
| CNAα antibody | | |
| epitope | | |
| Amino acid | MSEPKAIDPKLSTTDRVVKAVPFPPSHRLTAKEVFDN | 3 |
| sequence of UniPro | DGKPRVDILKAHLMKEGRLEESVALRIITEGASILRQEKNL | |
| Q08209 (exemplary | LDIDAPVTVCGDIHGQFFDLMKLFEVGGSPANTRYLFLGDY | |
| sequence of CNAα) | VDRGYFSIECVLYLWALKILYPKTLFLLRGNHECRHLTEYF | |
| SEQ ID NOs: 1 and | TFKQECKIKYSERVYDACMDAFDCLPLAALMNQQFLCVHGG | |
| 2 are shown in | LSPEINTLDDIRKLDRFKEPPAYGPMCDILWSDPLEDFGNE | |
| bold underline in | KTQEHFTHNTVRGCSYFYSYPAVCEFLQHNNLLSILRAHEA | |
| SEQ ID NO: 3. | QDAGYRMYRKSQTTGFPSLITIFSAPNYLDVYNNKAAVLKY | |
| | ENNVMNIRQFNCSPHPYWLPNFMDVFTWSLPFVGEKVTEML | |
| | VNVLNICSDDELGSEEDGFDGATAAARKEVIRNKIRAIGKM | |
| | ARVFSVLREESESVLTLKGLTPTGMLPSGVLSGGKQTLQSA | |
| | TVEAIEADEAIKGFSPQHKITSFEEAKGLDRINERMPPRRD | |
| | AMPSDANLNSINKALTSETNGTDSNGSNSSNIQ | |
| Unphosphorylated | PQHKITSFEEA | 4 |
| amino acid | | |
| sequence | | |
| corresponding to | | |
| SEQ ID NO:1 | | |
| Amino acid | PPHRICSFEEA | 5 |
| sequence segment | | |
| from human CNAβ | | |
| corresponding to | | |
| the amino acid | | |
| sequence segment | | |
| from CNAα of SEQ | | |
| ID NO: 1. | | |
| Amino acid | LQHKIRSFEEA | 6 |
| sequence segment | | |
| from human CNA Υ | | |
| corresponding to | | |
| the amino acid | | |
| sequence segment | | |
| from CNAα of SEQ | | |
| ID NO: 1. | | |
| Heavy chain | EVQLQQSGAELVRPGASVKLSCTASDFNIKDTYIHWLKQRP | 7 |
| variable region | EQGLEWIGRIDPANSNPKYDPNFQGKATITTDTSSNTAYLQ | |
| (VH) of | LSSLTSDDTAVYYCTRRDYYGSGSWFAYWGQGTLVTVSS | |
| phosphospecific | | |
| S469 CNAα antibody | | |
| (CDRs shown in | | |
| double underline) | | |
| Heavy chain | 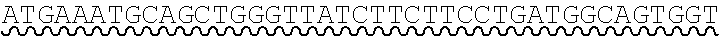 | 8 |
| variable region | 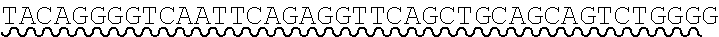 | |
| (VH) DNA | CAGAGCTTGTGAGGCCAGGGGCCTCAGTCAAGTTGTCCTGC | |
| (including leader | ACAGCTTCTGACTTCAACATTAAAGACACCTATATTCACTG | |
| sequence DNA) | GTTGAAGCAGAGGCCTGAACAGGGCCTGGAGTGGATTGGAA | |
| (CDRs shown in | GAATTGATCCTGCGAATAGTAATCCTAAATATGACCCGAAC | |
| double underline; | TTCCAGGGCAAGGCCACTATAACAACAGACACATCCTCCAA | |
| leader sequence in | CACAGCCTACCTGCAGCTCAGCAGCCTGACATCTGACGACA | |
| wavy underline) | CTGCCGTCTATTACTGTACTAGGAGGGATTACTACGGTAGT | |
| | GGTTCCTGGTTTGCTTACTGGGGCCAAGGGACTCTGGTCAC | |
| | TGTCTCTTCT | |
| Light chain | DIVMTQAAPSVPVTPGESVSISCRSSKSLLHSNGNTYLYWF | 9 |
| variable region | LQRPGQSPQLLIYRMSNLASGVPDRFSGSGSGTAFTLRINR | |
| (VH) of | VEAEDVGVYYCMQHLEYPLTFGAGTKLELK | |
| phosphospecific | | |
| S469 CNAα antibody | | |
| (CDRs shown in | | |
| double underline) | | |
| Light chain | 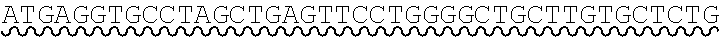 | 10 |
| variable region | 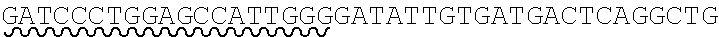 | |
| (VH) DNA | CACCCTCTGTACCTGTCACTCCTGGAGAGTCAGTATCCATC | |
| (including leader | TCCTGCAGGTCTAGTAAGAGTCTCCTGCATAGTAATGGCAA | |
| sequence DNA) | CACTTACTTGTATTGGTTCCTACAGAGGCCAGGCCAGTCTC | |
| (CDRs shown in | CTCAACTCCTGATATATCGGATGTCCAACCTTGCCTCAGGA | |
| double underline; | GTCCCAGACAGGTTCAGTGGCAGTGGGTCAGGAACTGCTTT | |
| leader sequence in | CACACTGAGAATCAATAGAGTGGAGGCTGAGGATGTGGGTG | |
| wavy underline) | TTTATTACTGTATGCAACATCTAGAATATCCGCTCACGTTC | |
| | GGTGCTGGGACCAAGCTGGAGCTGAAA | |
| Heavy chain CDR1 | DTYIH | 11 |
| (HC CDR1) of | | |
| phosphospecific | | |
| S469 CNAα antibody | | |
| HC CDR2 of | RIDPANSNPKYDPNFQG | 12 |
| phosphospecific | | |
| S469 CNAα antibody | | |
| HC CDR3 of | RDYYGSGSWFAY | 13 |
| phosphospecific | | |
| S469 CNAα antibody | | |
| Light chain CDR1 | RSSKSLLHSNGNTYLY | 14 |
| (LC CDR1) of | | |
| phosphospecific | | |
| S469 CNAα antibody | | |
| LC CDR2 of | RMSNLAS | 15 |
| phosphospecific | | |
| S469 CNAα antibody | | |
| LC CDR3 of | MQHLEYPLT | 16 |
| phosphospecific | | |
| S469 CNAα antibody | | |
| Heavy chain | MKCSWVIFFLMAVVTGVNS | 17 |
| variable region | | |
| leader sequence | | |
| Light chain | MRCLAEFLGLLVLWIPGAIG | 18 |
| variable region | | |
| leader sequence | | |

1. A method for measuring neuronal activity comprising:
a) obtaining a tissue sample comprising neurons or a sample comprising cultured neurons;
b) exposing the sample to one or more chemical compounds or physical signals that may alter activity of neurons in the sample;
c) exposing the sample to a phospho-S469 CNAα antibody; and
d) detecting the level of phospho-S469 CNAα antibody retained by the sample, wherein the level of phospho-S469 CNAα antibody retained by the sample indicates the neuronal activity level in the sample.

2. (canceled)

3. The method of claim 1, wherein the sample is exposed to a phospho-S469 CNAα antibody for 16-20 hours at 3-8° C. or for 1 to 2 hours at 20-30° C.

4. The method of claim 1, wherein the level of phospho-S469 CNAα antibody retained by the sample is detected by fluorescence or chemiluminescence staining

5. The method of claim 1, wherein the level of phospho-S469 CNAα antibody retained by the sample is detected in less than 30 minutes, less than 15 minutes, less than 10 minutes, or less than 5 minutes after exposure of the sample to the phospho-S469α antibody.

6. The method of claim 1, wherein the method comprises determining chemical long term potentiation (cLTP) of neurons in the sample from exposure to the one or more chemical compounds, wherein the level of phospho-S469 CNAα antibody retained by the sample indicates the cLTP.

7. The method of claim 1, wherein the sample comprises cultured neurons and the cultured neurons are derived from induced pluripotent stem cells (iPSCs) or from mammalian hippocampal, cortical, striatum, cerebellar granule, or other mammalian neuronal cells.

8. The method of claim 1, wherein the sample is a brain tissue sample.

9-26. (canceled)

27. The method of claim 1, wherein the phospho-S469 CNAα antibody comprises a heavy chain and a light chain, wherein the heavy chain comprises a heavy chain complementary determining region 1 (HCDR1) comprising the amino acid sequence of SEQ ID NO: 11; a HCDR2 comprising the amino acid sequence of SEQ ID NO: 12; and a HCDR3 comprising the amino acid sequence of SEQ ID NO: 13; and wherein the light chain comprises a light chain complementary determining region 1 (LCDR1) comprising the amino acid sequence of SEQ ID NO: 14; a LCDR2 comprising the amino acid sequence of SEQ ID NO: 15; and a LCDR3 comprising the amino acid sequence of SEQ ID NO: 16.

28. The method of claim 27, wherein the heavy chain of the antibody comprises a heavy chain variable region with an amino acid sequence that is at least 90%, at least 95%, at least 97%, at least 98%, or at least 99% identical to the amino acid sequence of SEQ ID NO: 7.

29. The method of claim 27, wherein the light chain of the antibody comprises a light chain variable region amino acid sequence that is at least 90%, at least 95%, at least 97%, at least 98%, or at least 99% identical to the amino acid sequence of SEQ ID NO: 9.

30. The method of claim 27, wherein the heavy chain variable region of the antibody comprises the amino acid sequence of SEQ ID NO: 7.

31-69. (canceled)


SEQUENCE LISTING (18 sequences)

SEQ ID NO: 1 - protein, 11 aa, Artificial Sequence
  Phosphospecific S469 CNA-alpha antibody epitope
  MOD_RES (7)..(7): Phosphorylated serine
PQHKITSFEEA

SEQ ID NO: 2 - protein, 16 aa, Artificial Sequence
  Pan CNA-alpha antibody epitope
RRDAMPSDANLNSINK

SEQ ID NO: 3 - protein, 521 aa, Homo sapiens
MSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEEDGFDGATAAARKEVIRNKIRAIGKMARVFSVLREESESVLTLKGLTPTGMLPSGVLSGGKQTLQSATVEAIEADEAIKGFSPQHKITSFEEAKGLDRINERMPPRRDAMPSDANLNSINKALTSETNGTDSNGSNSSNIQ

SEQ ID NO: 4 - protein, 11 aa, Artificial Sequence
  Unphosphorylated amino acid sequence corresponding to SEQ ID NO1
PQHKITSFEEA

SEQ ID NO: 5 - protein, 11 aa, Homo sapiens
PPHRICSFEEA

SEQ ID NO: 6 - protein, 11 aa, Homo sapiens
LQHKIRSFEEA

SEQ ID NO: 7 - protein, 121 aa, Artificial Sequence
  Heavy chain variable region (VH) of phosphospecific S469 CNA-alpha antibody
EVQLQQSGAELVRPGASVKLSCTASDFNIKDTYIHWLKQRPEQGLEWIGRIDPANSNPKYDPNFQGKATITTDTSSNTAYLQLSSLTSDDTAVYYCTRRDYYGSGSWFAYWGQGTLVTVSS

SEQ ID NO: 8 - DNA, 420 bp, Artificial Sequence
  Heavy chain variable region (VH) DNA (including leader sequence DNA)
atgaaatgcagctgggttatcttcttcctgatggcagtggttacaggggtcaattcagaggttcagctgcagcagtctggggcagagcttgtgaggccaggggcctcagtcaagttgtcctgcacagcttctgacttcaacattaaagacacctatattcactggttgaagcagaggcctgaacagggcctggagtggattggaagaattgatcctgcgaatagtaatcctaaatatgacccgaacttccagggcaaggccactataacaacagacacatcctccaacacagcctacctgcagctcagcagcctgacatctgacgacactgccgtctattactgtactaggagggattactacggtagtggttcctggtttgcttactggggccaagggactctggtcactgtctcttct

SEQ ID NO: 9 - protein, 112 aa, Artificial Sequence
  Light chain variable region (VH) of phosphospecific S469 CNA-alpha antibody
DIVMTQAAPSVPVTPGESVSISCRSSKSLLHSNGNTYLYWFLQRPGQSPQLLIYRMSNLASGVPDRFSGSGSGTAFTLRINRVEAEDVGVYYCMQHLEYPLTFGAGTKLELK

SEQ ID NO: 10 - DNA, 396 bp, Artificial Sequence
  Light chain variable region (VH) DNA (including leader sequence DNA)
atgaggtgcctagctgagttcctggggctgcttgtgctctggatccctggagccattggggatattgtgatgactcaggctgcaccctctgtacctgtcactcctggagagtcagtatccatctcctgcaggtctagtaagagtctcctgcatagtaatggcaacacttacttgtattggttcctacagaggccaggccagtctcctcaactcctgatatatcggatgtccaaccttgcctcaggagtcccagacaggttcagtggcagtgggtcaggaactgctttcacactgagaatcaatagagtggaggctgaggatgtgggtgtttattactgtatgcaacatctagaatatccgctcacgttcggtgctgggaccaagctggagctgaaa

SEQ ID NO: 11 - protein, 5 aa, Artificial Sequence
  Heavy chain CDR1 (HC CDR1) of phosphospecific S469 CNA-alpha antibody
DTYIH

SEQ ID NO: 12 - protein, 17 aa, Artificial Sequence
  Heavy chain CDR2 (HC CDR2) of phosphospecific S469 CNA-alpha antibody
RIDPANSNPKYDPNFQG

SEQ ID NO: 13 - protein, 12 aa, Artificial Sequence
  Heavy chain CDR3 (HC CDR3) of phosphospecific S469 CNA-alpha antibody
RDYYGSGSWFAY

SEQ ID NO: 14 - protein, 16 aa, Artificial Sequence
  Light chain CDR1 (LC CDR1) of phosphospecific S469 CNA-alpha antibody
RSSKSLLHSNGNTYLY

SEQ ID NO: 15 - protein, 7 aa, Artificial Sequence
  Light chain CDR2 (LC CDR2) of phosphospecific S469 CNA-alpha antibody
RMSNLAS

SEQ ID NO: 16 - protein, 9 aa, Artificial Sequence
  Light chain CDR3 (LC CDR3) of phosphospecific S469 CNA-alpha antibody
MQHLEYPLT

SEQ ID NO: 17 - protein, 19 aa, Artificial Sequence
  Heavy chain variable region leader sequence
MKCSWVIFFLMAVVTGVNS

SEQ ID NO: 18 - protein, 20 aa, Artificial Sequence
  Light chain variable region leader sequence
MRCLAEFLGLLVLWIPGAIG